(19) United States
(12) Patent Application Publication
(10) Pub. No.: US 2024/0158339 A1
(43) Pub. Date: May 16, 2024

(54) TYRAMINE CONTAINING HYDROXYCINNAMIC ACID AMIDE DERIVATIVES AND METHODS OF USE THEREOF

(71) Applicant: Brightseed, Inc., South San Francisco, CA (US)

(72) Inventors: Lee Heil Chae, San Francisco, CA (US); Jessica Leigh Ochoa, Half Moon Bay, CA (US); Dennis Solas, South San Francisco, CA (US)

(21) Appl. No.: 18/419,387

(22) Filed: Jan. 22, 2024

Related U.S. Application Data
(63) Continuation of application No. 16/510,005, filed on Jul. 12, 2019, now abandoned.
Application No. 16/510,005 is a continuation-in-part of application No. PCT/US2019/012986, filed on Jan. 10, 2019.
(60) Provisional application No. 62/615,615, filed on Jan. 10, 2018.

(51) Int. Cl. C07C 235/34; C07D 215/227; C07D 239/80

(52) U.S. Cl. CPC C07C 235/34; C07D 215/227; C07D 239/80

(57) ABSTRACT

Tyramine containing hydroxycinnamic acid amide derivatives are provided as are methods of using the same in modulating metabolism and addressing the underlying pathogenesis of metabolic disorders, such as nonalcoholic fatty liver disease, nonalcoholic steatohepatitis and type II diabetes mellitus.

### INTRODUCTION

This application is a continuation of U.S. application Ser. No. 16/510,005 filed Jul. 12, 2019, which is a continuation-in-part of U.S. Serial No. PCT/US2019/012986 filed Jan. 10, 2019, which claims the benefit of priority of U.S. Provisional Application No. 62/615,615 filed Jan. 10, 2018, the contents of which are incorporated herein by reference in their entireties.

### BACKGROUND

The “Western Diet” has been associated with a global rise in metabolic disorders such as obesity, type II diabetes mellitus (T2DM), metabolic syndrome, nonalcoholic fatty liver disease (NAFLD), heart disease, and stroke. Interactions between genetic and environmental factors such as diet and lifestyle, particularly over-nutrition and sedentary behavior, promote the progression and pathogenesis of these polygenic diet-related diseases. Their current prevalence is increasing dramatically to epidemic proportions. Nutrition is probably the most important environmental factor that modulates expression of genes involved in metabolic pathways and the variety of phenotypes associated with obesity, the metabolic syndrome, and type II diabetes mellitus. Furthermore, the health effects of nutrients may be modulated by genetic variants.

A 70% ethyl alcohol extract of Tribulus terrestris has been suggested to provide a protective effect in a model of type I diabetes mellitus (i.e., streptozotocin-induced diabetic rats) by inhibiting oxidative stress (Amin, et al. (2006) Ann. NY Acad. Sci. 1084:391-401).

U.S. Pat. Nos. 8,481,593 and 9,089,499 disclose para-coumaric acid derivatives such as N-trans-feruloyltyramine in topical and cosmetic compositions for use in inhibiting human tyrosinase and in the treatment of hyperpigmentation.

An acetone extract from Smilax aristolochiifolia root, which is enriched for N-trans-feruloyltyramine, has been suggested to be useful in counteracting some symptoms (e.g., hypertriglyceridemia, insulin resistance, blood pressure, and inflammation) in an injury model associated with metabolic syndrome (Amaro, et al. (2014) Molecules 19:11366-84).

US 2008/0132544 suggests the use of isolated N-trans-feruloyltyramine from Piper nigrum in a composition for the treatment of visceral fat obesity, T2DM, insulin resistant syndrome and metabolic syndrome.

### SUMMARY OF THE INVENTION

The present invention provides a compound having the structure of Formula I, or an isomer, salt, homodimer, heterodimer, or conjugate thereof:

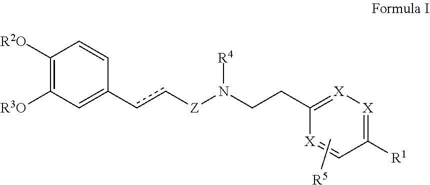

wherein
each occurrence of X is independently C or N;
Z is —CR⁶— or —SO₂—
R¹ is an —OH, —OCH₂CH₂R⁷, or —NHR⁸ group, or R¹ together with R⁵ form a 6-membered substituted heterocycloalkyl ring,
R² and R³ are independently a hydrogen or —CH₂ CH₂R₇ group, or R₂ and R₃ together form a five- or six-membered heterocycloalkyl ring;
R⁴ is a hydrogen or —CH₂CH₂R⁷ group;
R⁵ is present or absent and when present is a substituent on one or more ring atoms and for each occurrence is independently a halo, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl;
R⁶ is H₂, oxo, substituted alkyl, spirocycloalkyl or spiroheterocycloalkyl;
R⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl;
R⁸ is substituted sulfonyl, substituted alkyl, carboxyl ester or aminocarbonyl; and the dashed bond is present or absent.

In some embodiments, the compound has the structure of Formula II-IX:

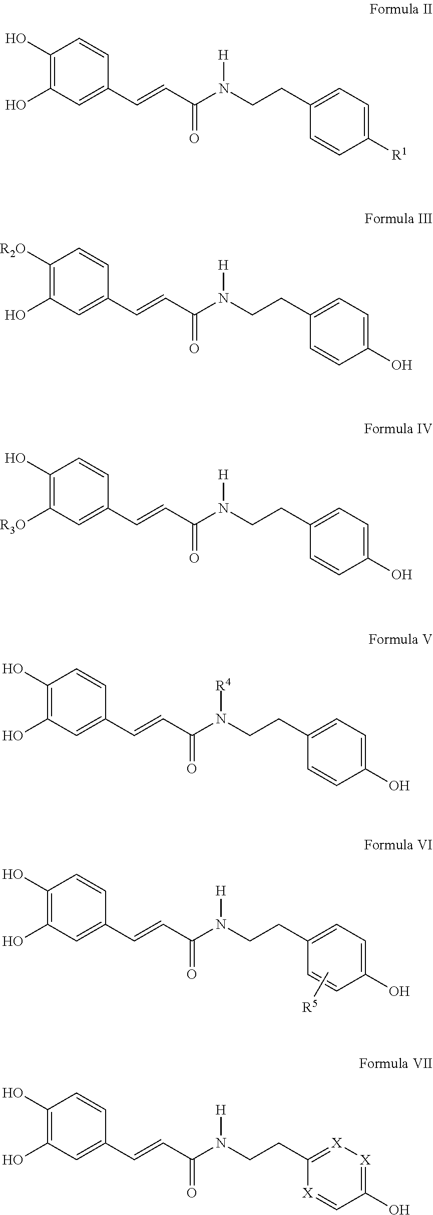

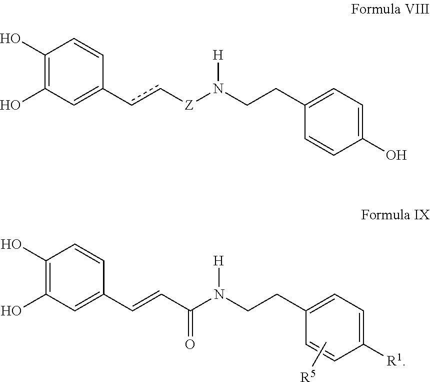

A dietary supplement, food ingredient or additive, a medical food, nutraceutical or pharmaceutical composition comprising the compound is also provided as are methods of using such compositions for modulating metabolism, e.g., in a subject having or at risk of developing a metabolic disorder.

### BRIEF DESCRIPTION OF THE DRAWINGS

FIG. 1 shows a dose-response analysis of N-trans-caffeoyltyramine, N-trans-feruloyltyramine and coumaroyltyramine in an assay measuring insulin promoter activity. Dimethylsulfoxide (DMSO) and alverine (20 μM) were used as negative and positive controls, respectively.

FIG. 2 shows the effect of N-trans-caffeoyltyramine, N-trans-feruloyltyramine and coumaroyltyramine on insulin mRNA levels as determined by quantitative PCR. DMSO and alverine (20 μM) were used as negative and positive controls, respectively.

FIG. 3 shows the effect of N-trans-caffeoyltyramine, N-trans-feruloyltyramine and coumaroyltyramine on HNF4α mRNA levels as determined by quantitative PCR. DMSO and alverine (20 μM) were used as negative and positive controls, respectively.

FIG. 4 shows that N-trans-caffeoyltyramine-mediated increases in insulin expression are inhibited by BI-6015, α known HNF4a antagonist.

FIG. 5 shows the effect of N-trans-caffeoyltyramine and N-trans-feruloyltyramine on estrogenic activity. Assays were carried out in the presence (1 μM) or absence (0 μM) Tamoxifen (Tam) using Alverine and 7005 (CAS No. 380336-903) (known HNF4α transcriptional activators) as positive controls and cis-feruloyltyramine and DMSO as negative controls.

FIG. 6 demonstrates that N-trans-caffeoyltyramine and N-trans-feruloyltyramine can reverse fat accumulation. T6PNE cells were pretreated for 1 day with 0.06 mM, 0.12 mM or 0.25 mM palmitate at which time 15 μM N-trans-caffeoyltyramine or control (DMSO) was added. Cells were harvested on day 3, 6 and 8 and subjected to staining with Nile Red and Oil Red P. Results are expressed as fold change in Nile Red staining: +palmitate/10% FBS medium (no palmitate).

FIG. 7 shows that N-trans-caffeoyltyramine increases nuclear expression of HNF4α in the liver.

FIG. 8 shows that lipid droplet size in the liver is reduced by treatment with N-trans-caffeoyltyramine.

FIG. 9 shows levels of blood analytes including alkaline phosphatase (ALP), alanine transaminase (ALT), y-glutamyltransferase(GGT), biliary artresia, total bilirubin, albumin, blood urea nitrogen (urea), and cholesterol in mice treated with N-trans-caffeoyltyramine or control (DMSO).

FIG. 10 shows triglyceride mice fed a high fat diet and treated with N-trans-caffeoyltyramine or control (DMSO).

FIG. 11 shows the effect of N-trans-caffeoyltyramine on HNF4α expression in the pancreas of mice fed a high fat diet as compared to control (DMS).

FIG. 12 shows the effect of N-trans-caffeoyltyramine on HNF4a expression in the intestine of mice fed a high fat diet as compared to control (DMSO).

FIG. 13 shows the amounts of N-trans-caffeoyltyramine, N-trans-feruloyltyramine and p-coumaroyltyramine present in ethanol extracts (% of extract, w/w) from a variety of sources including Tribulus terrestris seed (1), Cannabis (hemp) seed hull (2), Annona spp. (atemoya) seed (3), Annona muricata (Guanabana) seed (4), A. cherimola (Cherimoya) leaf (5), Zea mays (corn) stalk (6), Tribulus terrestris (Goat Head) seed (7), A. cherimola hardwood (bark and core) (8), Solanum lycopersicum ground pomace (9), S. tuberosum (yellow potato) peel (10), Piper nigrum (black peppercorn) fruit (11), S. tuberosum (purple potato) peel (12), S. tuberosum (red potato) peel (13), S. lycopersicum (tomato) pomace (14), S. lycopersicum (tomato) extruded pomace (15), A. muricata (Guanabana) leaves (16), Allium sativum (garlic) bulb (17), S. tuberosum (purple potato) peel (18), A. montana (Mountain soursop) leaves (19), Z. mays leaves (20), S. tuberosum (purple potato) sprouts (21), A. cherimola (Cherimoya) seed (22), Allium fistulosum (green onion) whole plant (23), S. tuberosum (white potato) peel (24), A. cherimola (Cherimoya) greenwood (25), Cannabis (hemp) leaves (26), S. tuberosum (white potato) peel (27), S. lycopersicum seed (28), S. lycopersicum (Beefsteak) whole fruit (29), A. muricata (Guarabana) skin of unripe fruit (30), A. muricata (Guanabana) ripe fresh fruit (31), A. squamosa (sweetsop) whole fruit (32), Capsicum annuum (serrano pepper) fruit (33), S. tuberosum (Russet potato) peel (34), Lycium barbarum (goji/wolf berry) fruit (35), S. tuberosum (purple potato) core (36), Chenopodium quinoa (quinoa) seed (37), Ipomoea batatas (sweet potato) whole potato (38), Ipomoea batatas (sweet potato) peel (39), Armoracia rusticana (horseradish) root (40), S. tuberosum (Colorado potato) peel (41), Fagopyrum esculentum (buckwheat) hulls (42), Capsicum frutescens (piri piri pepper) fruit (43), S. tuberosum (purple potato) core (44), C. annuum (Thai chili) stems and leaves (45), A. muricata (Guanabana) unripe fruit flesh (46), S. tuberosum (yellow potato) core (47), and Eragrostis tef (teff) seed (48).

### DETAILED DESCRIPTION OF THE INVENTION

This invention provides tyramine containing hydroxycinnamic acid amide derivatives of use in modulating metabolism, in particular HNF4a activity, thereby mitigating the adverse effects of free fatty acids in both liver cells and pancreatic ˜-cells. The tyramine containing hydroxycinnamic acid amide derivatives of this invention are analogs of lead compounds identified in traditional screening assays for agents that modulate known signaling pathways Similar to tyramine containing hydroxycinnamic acid amides the derivatives herein are expected to exhibit dose-response HNF4α activity and upregulate insulin gene expression. Further, these derivatives are expected to show strong, lipid clearing activity in a hepatocyte (hepG2) lipid challenge model of fatty liver disease. Genetic, functional genomic, transcriptomic and clinical evidence indicate that HNF4a agonists can improve overall metabolic health by enabling the body to maintain sugar and lipid homeostasis. Accordingly, the derivatives herein are of use in methods of promoting and/or recovering healthy HNF4α function, mitigating the adverse effects of free fatty acids, modulating metabolism, and addressing the underlying pathogenesis of metabolic disorders, such as NAFLD, nonalcoholic steatohepatitis (NASH) and T2DM. Using the composition of this invention, health and well-being are improved and promoted.

### Derivatives

This invention provides tyramine containing hydroxycinnamic acid amide derivatives or analogs, and their use in modulating metabolism. As used herein, the term “tyramine containing hydroxycinnamic acid amide derivative” (or “tyramine containing hydroxycinnamic acid amide analog”) refers to a compound having the structure of Formula I, or an isomer, salt, homodimer, heterodimer, or conjugate thereof:

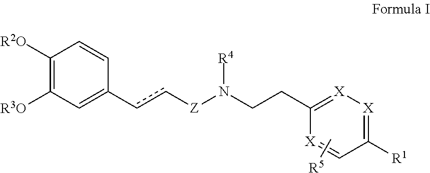

wherein
each occurrence of X is independently C or N;
Z is —CR ⁶— or —SO₂—
R¹ is an —OH, —OCH₂CH₂R⁷, or —NHR⁸ group, or R¹ together with R⁵ form a 6-membered substituted heterocycloalkyl ring,
R² and R³ are independently a hydrogen or —CH₂ CH₂ R⁷ group, or R² and R³ together form a five- or six-membered heterocycloalkyl ring;
R⁴ is a hydrogen or —CH₂ CH₂R⁷ group;
R⁵ is present or absent and when present is a substituent on one or more ring atoms and for each occurrence is independently a halo, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl;
R⁶ is H₂, oxo, substituted alkyl, spirocycloalkyl or spiroheterocycloalkyl;
R⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl;
R⁸ is substituted sulfonyl, substituted alkyl, carboxyl ester or aminocarbonyl; and
the dashed bond is present or absent.

“Alkyl” refers to monovalent saturated aliphatic hydrocarbyl groups having from 1 to 10 carbon atoms and, in some embodiments, from 1 to 6 carbon atoms. Alkyl groups including subscripted numbers, e.g., “(Cn”),” further define the groups by providing the exact number (n) of carbon atoms in the group. For example, “C1-C6-alkyl” designates those alkyl groups having from 1 to 6 carbon atoms (e.g., 1, 2, 3, 4, 5, or 6, or any range derivable therein (e.g., 3-6 carbon atoms)). “Lower alkyl” is intended to mean a branched or unbranched saturated monovalent hydrocarbon radical containing 1 to 6 carbon atoms (i.e., C₁-C₆-alkyl). Alkyl includes, by way of example, linear and branched hydocarbyl groups such as methyl (—CH₃), ethyl (—CH2CH₃), n-propyl (—CH2CH2CH₃), isopropyl (—CH(CH₃)2), n-butyl (—CH₂CH₂CH₂CH₃), isobutyl (—CHCH₂ (CH₃)₂), sec-butyl (—CH(CH₂CH₃)(CH₃)), t-butyl (—C(CH₃)₃), and n-pentyl (—CH₂CH₂CH₂CH₂CH₃).

“Substituted alkyl” refers to an alkyl group having from 1 to 5 and, in some embodiments, 1 to 3 or 1 to 2 “substituents” as defined herein.

“Alkoxy” refers to the group -0-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O— (substituted alkyl) wherein substituted alkyl is as defined herein.

“Amino” refers to the group —NH₂.

“Substituted amino” refers to the group —NR⁹K¹⁰ where R⁹ and R are independently selected from hydrogen or the “substituents” as defined herein. When R⁹ is hydrogen and R¹⁰ is alkyl, the substituted amino group is sometimes referred to herein as alkylamino. When R⁹ and R¹⁰ are alkyl, the substituted amino group is sometimes referred to herein as dialkylamino When referring to a monosubstituted amino, it is meant that either R⁹ or R¹⁰ is hydrogen but not both. When referring to a disubstituted amino, it is meant that neither R⁹ nor R¹⁰ are hydrogen.

“Aminocarbonyl” refers to the group —C(=0) NR¹¹ R¹² where R¹¹ and R¹² are independently selected from the group of hydrogen, alkyl, or substituted alkyl as defined herein.

As used herein, the term “amido” refers to aminocarbonyl- or carbamoyl- or —NH (C═O) R¹³,wherein R¹³ is selected from the “substituents” as defined herein.

“Aryl” refers to an aromatic group of from 6 to 14 carbon atoms and no ring heteroatoms and having a single ring (e.g. , phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom.

“Substituted aryl” refers to aryl groups which are substituted with 1 to 8 and, in some embodiments, 1 to 5, 1 to 3, or 1 to 2 substituents selected from the group of “substituents” as defined herein.

“Cyano” or “carbonitrile” refers to the group —CN.

“Carbonyl” refers to the divalent group -c (0)- which is equivalent to —C(═O)—.

“Carboxyl” or “carboxy” refers to —COOH or salts thereof.

“Carboxyl ester” or “carboxy ester” refers to the groups —C(O)O-alkyl or —C(O)O-substituted alkyl, wherein alkyl and substituted alkyl are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. Examples of cycloalkyl groups include, for instance, adamantyl, cyclopropyl, cyclobutyl, cyclopentyl, cyclooctyl, and cyclohexenyl. “Cn-cycloalkyl” refers to cycloalkyl groups having n carbon atoms as ring members.

Substituted cycloalkyl” refers to a cycloalkyl group, as defined herein, having from 1 to 8, or 1 to 5, or in some embodiments 1 to 3 “substituents” as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 14 carbon atoms and 1 to 6 heteroatoms selected from the group of oxygen, nitrogen, and sulfur and includes a 5-to 18-member ring or ring system that includes a single ring (e.g., imidazolyl) or multiple rings (e.g., benzimidazol-2-yl and benzimidazol-6-yl). In certainembodiments, the heteroaryl a 4- to 6-member ring with 1 to 4 heteroatoms. For multiple ring systems, including fused, bridged, and spiro ring systems having aromatic and non-aromatic rings, the term “heteroaryl” applies if there is at least one ring heteroatom and the point of attachment is at an atom of an aromatic ring. More specifically the term heteroaryl includes, but its not limited to, pyridyl, furanyl, thienyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl,isoquinolyl, quinazolinonyl, benzimidazolyl, benzisoxazolyl, or benzothienyl.

“Substituted heteroaryl” refers to a heteroaryl group that is substituted with from 1 to 8, or in some embodiments 1 to 5, or 1 selected from the group herein.

“Heterocycle” or “heterocyclic” or “heterocyclo” or “heterocycloalkyl” or “heterocyclyl” refers to a saturated or partially saturated cyclic group having from 1 to 14 carbon atoms and from 1 to 6 heteroatoms selected from the group of nitrogen, sulfur, or oxygen and includes single ring and multiple ring systems including fused, bridged, and spiro ring systems. For multiple ring systems having aromatic and/or non-aromatic rings, the term “heterocyclic”, “heterocycle”, “heterocyclo”, “heterocycloalkyl” or “heterocyclyl” applies when there is at least one ring heteroatom and the point of attachment is at an atom of a non-aromatic ring. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., Cr C10) refers to the total number of carbon atoms in the portion of the heterocyclyl group exclusive of the number of heteroatoms.

“Substituted heterocycle” or “substituted heterocyclic” or “substituted heterocyclo” or “substituted heterocycloalkyl” or “substituted heterocyclyl” refers to heterocyclic groups, as defined herein, that are substituted with from 1 to 5 or in some embodiments 1 to 3 of the “substituents” as defined herein.

“Oxo” refers to the atom (═O).

“Spirocycloalkyl” refers to a 3- to 10-member cyclic substituent formed by replacement of two hydrogen atoms at a common carbon atom with a cycloalkyl group having 2 to 9 carbon atoms, as exemplified by the following structure wherein the carbon attached to bonds marked with wavy lines is substituted with a spirocycloalkyl group:

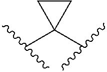 [SMILES: *C1(*)CC1]

“Spiroheterocycloalkyl” refers to a 3- to 10-member heterocyclic substituent formed by replacement of two hydrogen atoms at a common carbon atom with a heterocycloalkyl group having 2 to 9 carbon atoms, as exemplified by the following structure wherein the carbon attached to bonds marked with wavy lines is substituted with a spiroheterocycloalkyl group:

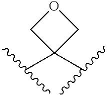 [SMILES: *C1(*)COC1]

“Sulfonyl” refers to the divalent group —S(O)₂—.

Unless otherwise defined, a “substituent” refers to a group selected from alkyl, substituted alkyl, alkoxy, substituted alkoxy, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic acyl, acylamino, acyloxy, amino, substituted amino, aminocarbonyl, aminothiocarbonyl, aminocarbonylamino, aminothiocarbonylamino, aminocarbonyloxy, amino s ulfonyl, amino sulfonyloxy , aminosulfonylamino, amidino, aryloxy, substituted aryloxy, arylthio, substituted arylthio, azido, carboxyl, carboxyl ester, (carboxyl ester)amino, (carboxyl ester)oxy, cyano, cycloalkyloxy, substituted cycloalkyloxy, cycloalkylthio, substituted cycloalkylthio,guanidino, substituted guanidino, halo, hydroxy, oxo, hydroxyamino, alkoxyamino, hydrazino, substituted hydrazino, heteroaryloxy, substituted heteroaryloxy, heteroarylthio, substituted heteroarylthio, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio.

Any undefined valency on an atom of a structure shown in this application implicitly represents a hydrogen atom bonded to the atom.

In some embodiments, the tyramine containing hydroxycinnamic acid amide derivative is a compound having the structure of Formula II, or an isomer, salt, homodimer, heterodimer, or conjugate thereof:

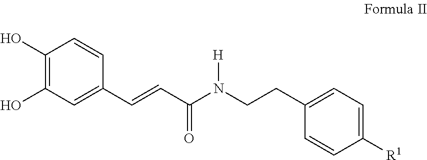

wherein
R¹ is —OCH₂ CH₂R⁷, and
R⁷ is a hydrogen, hydroxy, alkyl, substituted alkyl, alkoxy, substituted sulfonyl, carboxyl ester, aminosubstituted amino, cyano, aryl, substituted aryl, cycloalkyl, heteroaryl, substituted heteroaryl.

In other embodiments, the tyramine containing hydroxycinnamic acid amide derivative is a compound having the structure of Formula III, or an isomer, salt, homodimer, heterodimer, or conjugate thereof:

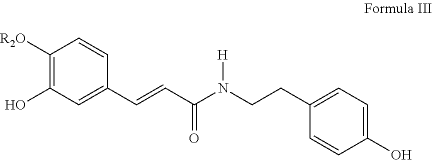

wherein
R² is —CH₂CH₂R⁷, and
R⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl.

In further embodiments, the tyramine containing hydroxycinnamic acid amide derivative is a compound having the structure of Formula IV, or an isomer, salt, homodimer, heterodimer, or conjugate thereof:

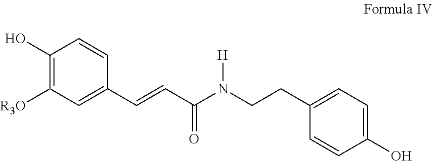

wherein
R³ is —CH₂CH₂R⁷, and
R⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl.

In yet other embodiments, the tyramine containing hydroxycinnamic acid amide derivative is a compound having the structure of Formula V, or an isomer, salt, homodimer, heterodimer, or conjugate thereof:

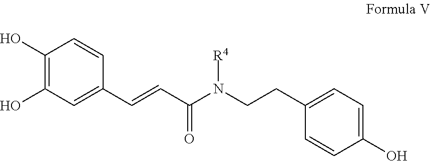

wherein
R⁴ is —CH₂CH₂R⁷, and
R ⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl.

In yet another embodiment, the tyramine containing hydroxycinnamic acid amide derivative is a compound having the structure of Formula V, or an isomer, salt, homodimer, heterodimer, or conjugate thereof:

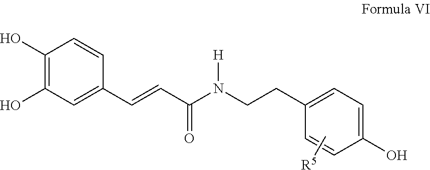

wherein
R⁵ is a substituent on one or more ring atoms (e.g., position 2 and/or 3) and is for each occurrence independently a halo, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl;

In still a further embodiment, the tyramine containing hydroxycinnamic acid amide derivative is a compound having the structure of Formula VII, or an isomer, salt, homodimer, heterodimer, or conjugate thereof:

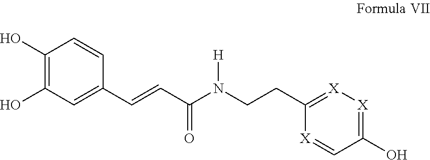

wherein each occurrence of X is independently C or N. In certain embodiments, at least one X is N.

In a still further embodiment, the tyramine containing hydroxycinnamic acid amide derivative is a compound having the structure of Formula VIII, or an isomer, salt, homodimer, heterodimer, or conjugate thereof:

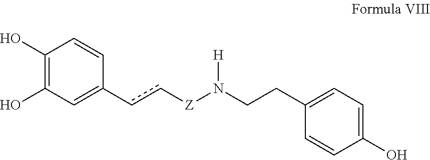

wherein
Z is —CR⁶— or —SO₂—;
R⁶ is H₂, is substituted alkyl, spirocycloalkyl or spiroheterocycloalkyl; and the dashed bond is present or absent.

In a further embodiment, the tyramine containing hydroxycinnamic acid amide derivative is a compound having the structure of Formula IX, or an isomer, salt, homodimer, heterodimer, or conjugate thereof:

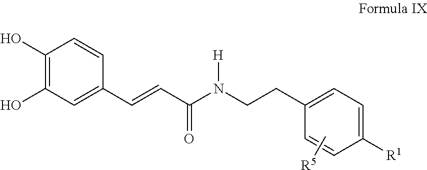

wherein
R¹ is an —NHR⁸ group, or R¹ together with R⁵ form a 6-membered substituted heterocycloalkyl ring;
R⁸ is substituted sulfonyl, substituted alkyl, carboxyl ester or aminocarbonyl.

“Isomer” refers to especially optical isomers (for example essentially pure enantiomers, diastereomers, and mixtures thereof) essentially pure as well as conformation isomers (i.e., isomers that differ only in their angles of at least one chemical bond), position isomers (particularly tautomers), and geometric isomers (e.g., cis-trans isomers).

A “salt” of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like i or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methane sulfonic acid, ethane sulfonic acid 1,2-ethane-disulfonic acid, 2-hydroxyethane sulfonic acid, benzene sulfonic acid, 4 -chlorobenzenesulfonic acid, 2naphthalenesulfonic acid 4-toluenesulfonic camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

A homodimer is a molecule composed of two identical tyramine containing hydroxycinnamic acid amide derivative subunits. By comparison, a heterodimer is a molecule composed of two different tyramine containing hydroxycinnamic acid amide derivative subunits. Examples of known homodimers of the parent compounds include a cross-linked N-trans-feruloyltyramine dimer. See, for example, King & Calhoun (2005) Phytochemistry 66(20):2468-73. Conjugates of monomers of tyramine containing hydroxycinnamic acid amide derivatives and other compounds, such as lignan amides is also contemplated. Examples of conjugates include, but are not limited to, derivatives of cannabisin A, cannabisin B, cannabisin C, cannabisin D, cannabisin E and cannabisin F.

The tyramine containing hydroxycinnamic acid amide derivatives may be synthesized by the methods exemplified herein or using other similar approaches. Once produced, the derivatives can be isolated and purified, e.g., by chromatography and/or crystallization to obtain high purity tyramine containing hydroxycinnamic acid amide derivatives. The solubility of a tyramine containing hydroxycinnamic acid amide derivative may be adjusted by changing temperature and/or the composition of the solution, for instance by removing ethanol, and/or adjusting the pH to facilitate precipitation, followed by filtration or centrifugation of the precipitated crystals or oils. Other suitable methods include, but are not limited to, liquid- liquid extraction, centrifugal partition chromatography or adsorption onto a resin or removal of impurities with resin.

A “substantially pure” preparation of a compound is defined as a preparation having a chromatographic purity (of the desired compound) of greater than 95%, more preferably greater than 96%, more preferably greater than 97%, more preferably greater than 98%, more preferably greater than 99% and most preferably greater than 99.5%, as determined by area normalization of an HPLC profile.

In certain embodiments of this invention, the compound is not one or more of the following.

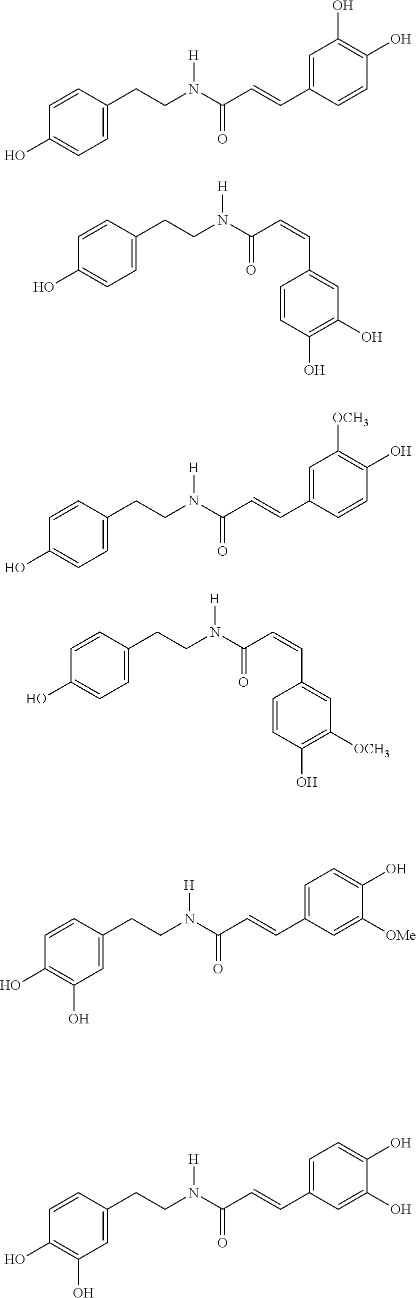 [SMILES: O=C(/C=C/c1ccc(O)c(O)c1)NCCc1ccc(O)cc1 ; O=C(/C=C\c1ccc(O)c(O)c1)NCCc1ccc(O)cc1 ; COc1cc(/C=C/C(=O)NCCc2ccc(O)cc2)ccc1O ; COc1cc(/C=C\C(=O)NCCc2ccc(O)cc2)ccc1O ; COc1cc(/C=C/C(=O)NCCc2ccc(O)c(O)c2)ccc1O ; O=C(/C=C/c1ccc(O)c(O)c1)NCCc1ccc(O)c(O)c1]

### Biological Activity

Biological activity of tyramine containing hydroxycinnamic acid amide derivatives can be determined using one or more of the well-known biological in vitro assays, in vivo assays and animal models described in more detail below. Each of these assays would provide a measure of the activity of the derivatives of the instant invention to provide beneficial effects on cellular endpoints linked to metabolic disorders including but not limited to obesity, T2DM, heart disease, stroke, fatty liver disease (NAFLD) and nonalcoholic steatohepatitis (NASH).

Triglyceride Assay in Cultured Hepatocytes. For measuring triglyceride synthesis in cultured primary hepatocytes, freshly isolated hepatocytes (e.g., from rats) are cultured for 24 hours with normal media (Dulbecco's modified Eagle Medium (DMEM) with 0.25% bovine serum albumin) in the presence or absence of monounsaturated and/or saturated fatty acids (e.g., palmitate (C16:0) or oleate (C18:1), or a 2:1 mixture of the two) and presence or absence of a compound of the invention. Quantitative estimation of hepatic triglyceride accumulation is performed by extraction of hepatic lipids from cell homogenates using chloroform/methanol (2:1) and enzymatic assay of triglyceride mass using an ENZYCHROMTM Triglyceride Assay Kit (Bioassay Systems, Hayward, CA).

Adipocyte Glucose Consumption Equal amounts (5×10⁵ cells) of 3T3-L1 pre-adipocytes are seeded and cultured in normal D-glucose, DMEM with 10% fetal bovine serum (FBS), penicillin-streptomycin in a humidified atmosphere of 95% air and 5% CO₂at 37° C. When the cells reach 100% confluence, 3T3-L1 pre-adipocytes are induced to be differentiate by treating the culture with 450 mg/dL D-glucose, 0.32 μM insulin, 0.5 mm 3-isobutyl-1-methylxanthine and 1 μM dexamethasone for 2 days. Subsequently, the culture medium of the differentiated adipocytes is changed to DMEM containing 450 mg/dL D-glucose with or without the administration of a compound of the invention. After 24 hours, the glucose consumption activity is determined by measuring the medium glucose concentration with insulin used as the positive control. Protocols and assays for glucose uptake into cells are available commercially (e.g., ABCAM; Cambridge, MA; Promega: Madison, WI).

Insulin Secretory Activity. Insulin-secreting cells, e.g., rat RIN-m5F cells, are plated into 96-well plates and used at subconfluence after a 24-hour incubation. Cells are exposed to 100 μl of sub-toxic concentrations of a compound of the invention and incubated at 37° C. with 5% CO₂ for 3 hours. Following treatment, plates are centrifuged at 1000 g for 10 minutes and insulin concentration of supernatants is determined using a solid phase two-site enzyme immunoassay, e.g., DRG Ultrasensitive Rat Insulin ELISA kit (DRG International, Inc.).

Insulin Promoter Activity. T6PNE cells (Kiselyuk, et al. (2012) Chem. Biol. 19(7):806-818; Kiselyuk, et al. (2010) J. Biomol. Screen 15(6):663-70) are seeded at 2000 cells per well in 384-well tissue culture plates in the presence of 1 μM tamoxifen and 0.03 mM palmitate. After a 24-hour incubation, a compound of the invention is added to the cells. Forty-eight hours after compound addition, cells are fixed in 4% paraformaldehyde and stained with DAPI. Blue (DAPI) and green (human insulin promoter driving GFP) channels are imaged.

Triglyceride Assay in Liver. Mice are provided a compound of the invention. Liver extracts are prepared by homogenization in 0.25% sucrose with 1 mmol/L EDTA, and lipids are extracted using chloroform/methanol (2:1 v/v) and suspended with 5% fatty acid-free bovine serum albumin. Triglyceride levels are measured using triglyceride assay reagents (Sigma Chemical Co.).

Hepatic Triglyceride Secretion in vivo. This assay employs the use of TRITON WR1339, which inhibits all lipoprotein lipases and therefore clearance of triglycerides from the blood (Millar et al. 2005. J. Lipid Res. 46:2023-2028). Mice are provided a compound of the invention. Subsequently, the mice are injected with 10% TRITON WR1339 per animal by intravenous (IV) injection and blood is collected to assess triglycerides at 0 minutes, 1 hour and 2 hours. Plasma is separated and assayed for triglycerides. Triglyceride secretion rates are expressed as milligram per kilogram per hour after normalizing with their liver weight.

De Novo Lipogenesis Assay. De Novo lipogenesis is thought to be involved in the pathogenesis of NAFLD (Sanders and Griffin. 2016. Biol. Rev. Camb. Philos. Soc. 91(2):452-468). Primary hepatocytes from animals treated with a compound of the invention are cultured overnight with 10% DMEM containing insulin (100 nM) and dexamethasone (1 μM). Cells are subsequently incubated with 74 KBq/m1 (2-14 ° C.) sodium acetate (2.07 GBq/mmol) for 1 hour. The cells are lysed with 1 N NaOH, acidified, and lipids are extracted with petroleum ether. Radioactivity is measured by liquid scintillation counter.

Animal Models of T2DM. Models of T2DM include but are not limited to leptin-deficient mouse (ob/ob; Drel, et al. (2006) Diabetes 55(12):3335-43; Wang, et al. (2014) Curr. Diabetes Rev. 10(2):131-145), the leptin-receptor- deficient mouse (db/db; Wang, et al. (2014) Curr. Diabetes Rev. 10(2):131-145), the obese Zucker rat (fa/fa; Shiota & Printz (2012) Methods Mol. Biol. 933:103-23) , the Wistar Kyoto rat (fa/fa; Figlewicz, et al. (1986) Peptides 7:61-65), proopiomelanocortin-deficient mice (POMc−/−; Yaswen, et al. (1999)Nat. Med. 5:1066-1070), melanocortin 3 and 4 receptor knockout animals (Huszar, et al. (1997) Cell 88:131-141; Butler, et al. (2000) Endocrinology 141(9):3518-21; Mul, et al. (2011) Obesity (Silver Spring) 20(3):612-21; Chen, et al. (2000) Nat. Genet. 26(1):97-102), animals overexpressing glucose transporter subtype 4 (Shepard, et al. (1993) J. Biol. Chem. 268:22243-22246) and neuron-specific insulin receptor knockout mice (NIRKO mice; Bruning, et al. (2000) Science 289:2122-2125). Reviews of the use of such animal models are available (e.g., Chatzigeorgiou et al. 2009. In Vivo 28:345-258; King, A.J.K. 2012. Br. J. Pharmacol. 166:877-894). These models are characterized by insulin resistance, hyperglycemia, and hyperinsulinemia, symptoms mirrored in human T2DM. Animals are provided with a compound of the invention and maximum tolerated dose and improvements in metabolism are evaluated.

Animal Model of Lipodystrophy. The complete lack of fat tissue (lipodystrophy) leads to similar metabolic changes as severe obesity and is associated with insulin resistance. Genetically modified mice with a lack of adipose tissue are characterized by hyperphagia, hepatic steatosis, hypertriglyceridaemia, insulin resistance and T2DM (Savage (2009) Dis. Model Mech. 2(11-12) :554-62). Due to the lack of functional adipose tissue, these mice are leptin deficient and are of use in assessing the effect of the compound of this invention on dysregulated metabolism. Such models are useful for demonstrating in vivo response for compounds of the present invention and exploring key concepts such as dose-response.

Rat Models of Diet-Induced Obesity. Outbred Sprague-Dawley rats have been used as a polygenic model of obesity (Levin, et al. (1997) Am. J. Physiol. 273:R725-30). Similarly, rats offered a varied and palatable diet which mimics the so-called Western diet of humans (cafeteria diet) become obese due to hyperphagia (Rogers & Blundell (1984) Neurosci. Biobehay. Rev. 8(4):441-53) Likewise, animals exposed to high-fat (HF) diets develop obesity and exhibit reductions in insulin and leptin sensitivity (Clegg, et al. (2011) Physiol. Behay. 103(1):10-6; Hariri & Thibault (2010) Nutr. Res. Rev. 23(2):270-99). Such models are useful for demonstrating in vivo response for compounds of the present invention and exploring key concepts such as dose-response.

Mouse Models of Diet-Induced Obesity. Diet-induced obese (DIO) mice are the standard to study lipotoxicity in vivo (Kennedy, et al. (2010) Disease Models & Mechanisms 3(3-4):156-66). High fat fed mice develop abnormalities in both the liver and pancreas. Depending on the genetic background, they develop insulin resistance with or without (3-cell atrophy and overt diabetes when on a high-fat diet (Leiter & Reifsnyder (2004) Diabetes 53 Suppl. 1:S4-11; Tschop & Heiman (2001) Exp. Clin. Endocrinol. Diabetes 109(6):307-19). Strains of mice that differ in propensity to develop -cell atrophy include, e.g., NONcNZ010/L0 (The Jackson Laboratory, Bar Harbor, ME) that develops -cell atrophy and C57BL/6J (The Jackson Laboratory, Bar Harbor, ME) that does not exhibit -cell loss. Using these models, the effect of normal vs. high fat diet±test compound can be analyzed. Approximately half of NONcNZ010/L0 males become diabetic and often develop islet atrophy on a high fat diet (Leiter (2009) Methods Mol. Biol. 560:1-17). Other strains that may be studied include the DIO mouse on the C57B1/6 background which is not highly prone to -cell loss but is a good model of pre-T2D and obesity with elevated blood glucose and impaired glucose tolerance (Leiter (2009) Methods Mol. Biol. 560:1-17). C57B1/6KsJ db/db mice develop diabetes associated with (3-cell failure (Hummel, et al. (1972) Biochem. Genet. 7(1):1-13), which has been shown to be correctable by MafA overexpression (Matsuoka, et al. (2015) J. Biol. Chem. 290:7647-7657), suggesting their use in an efficacy trial. Such models are useful for demonstrating in vivo response for compounds of the present invention and exploring key concepts such as dose-response.

Animal Model of Metabolic Syndrome. New Zealand obese (NZO) mouse are obese and have severe T2DM. A number of genetic susceptibility loci that favor the development of adiposity and hyperglycemia have been identified in NZO mice. In addition to the leptin receptor gene, several genes of transcription factors were identified as potential candidate genes and orthologs of some of these genes have been linked to the human metabolic syndrome (Joost (2010) Results Probl. Cell Differ. 520:1-11). Such models are useful for demonstrating in vivo response for compounds of the present invention and exploring key concepts such as dose-response.

Counter Screens. Counter screens are often used to select among a library of compounds in order to avoid off target effects. In the present invention, the activity of compounds as modulators of HNF4α activity is the desired target even though other off target effects may occur. Drugs that have been marketed for use in humans based on target effects other than HNF4a have subsequently been shown to have activity as HNF4α activators (Alverine and Benfluorex; Lee, et al. (2013) ACS Chem. Biol. 8(8):1730-6). Alverine has been marketed as a smooth muscle relaxant for gastrointestinal disorders, while Benfluorex was marketed as an anorectic agent. Benfluorex was known to be metabolized by cleavage of an ester moiety into fenfluramine, a potent agonist of serotonin 5-hydroxytryptamine 2 (5-HT₂ receptors, an effect that was thought to be related to its activity as an anorectic agent (Porter, et al. (1999) Br. J. Pharmacol. 128(1):13-20). However, modulation of 5-HT_{2A} receptors by Benfluorex was linked to undesirable cardiopulmonary side effects. Accordingly, based on these experiences with synthetic compounds, compounds of the present invention will be tested for off target effects on 5-hydroxytryptamine receptor activation using, e.g. a fluorometric imaging plate reader (FLIPR) assay, which allows rapid detection of rises in intracellular calcium levels in cells expressing a human 5-HT_{2A}, 5-HT_{2B} or 5-HT_{2c} receptor in CHO—Kl cells. See, e.g., Porter, et al. (1999) Br. J. Pharmacol. 128(1):13-20. Other counter screens may be chosen based on initial in vivo studies where toxic effects may be linked to other off target actions.

### Formulations

A substantially pure compound of this invention can be combined with a carrier and provided in any suitable form for consumption by or administration to a subject. In this respect, the compound is added as an exogenous ingredient or additive to the consumable. Suitable consumable forms include, but are not limited to, a dietary supplement, food ingredient or additive, a medical food, nutraceutical or pharmaceutical composition.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this invention can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food.

A dietary supplement is a product taken by mouth that contains a compound of the invention and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals Dietary supplements, nutraceuticals and pharmaceutical compositions can be found in many forms such as tablets, coated tablets, pills, capsules, pellets, granules, softgels, gelcaps, powders, emulsions, suspensions, elixirs, syrup, liquids, and any other form suitable for use.

The term “carrier” as used herein means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or body. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in conventional formulations.

In particular embodiments of this invention, a composition includes the compound, a carrier and a preservative to reduce or retard microbial growth. The preservative is added in amounts up to about 5%, preferably from about o. 01% to 1% by weight of the film. Preferred preservatives include sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA).

For preparing solid compositions such as tablets or capsules, the compound is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present invention. The tablets or pills containing the compound can be coated or otherwise compounded to provide a dosage form affording the advantage of prolonged action by sustained release of the active compound from the solid matrix and/or potentially enhanced absorption.

The liquid forms in which the compound of the invention is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this invention include the step of bringing into association a compound of carrier and, optionally, active ingredients. In prepared by uniformly the present invention with the one or more accessory and/or general, the formulations are and intimately bringing into association a compound of the present invention with liquid carriers, or finely divided solid carriers, or both, and then, if necessary, shaping the product. As such, the disclosed formulation may consist of, or consist essentially of a compound described herein in combination with a suitable carrier.

When a compound of the present invention is administered as pharmaceuticals, nutraceuticals, or dietary supplements to humans and animals, they can be given per se or as a composition containing, for example, 0.1 to 99% (more preferably, 10 to 30%) of active ingredient in combination with an acceptable carrier.

A composition of this invention may be administered to a subject to provide less than 100 mg of a compound disclosed herein per day. In certain embodiments, the consumable provides between 10 and 60 mg/day of a tyramine containing hydroxycinnamic acid amide derivative. The effective amount can be established by methods known in the art studies and be dependent upon bioavailability, toxicity, etc.

While it is contemplated that individual tyramine containing hydroxycinnamic acid amide derivatives may be used in the compositions of this invention, it is further contemplated that two or more of the derivative compounds could be combined in any relative amounts to produce custom combinations of ingredients containing two or more tyramine containing hydroxycinnamic acid amide derivatives in desired ratios to enhance product efficacy, improve organoleptic properties or some other measure of quality important to the ultimate use of the product.

### Molecular Target

HNF4α (hepatocyte nuclear factor 4α) is a global nuclear transcription factor, regulating expression of many genes involved in maintaining balanced metabolism (homeostasis). Notably, HNF4α is expressed in both the liver (hepatocytes) and pancreas (β-cells). The expression and transcriptional activity of HNF4α is decreased in NAFLD and T2DM in both human liver cells and human pancreatic β-cells. HNF4a is mutated in MODY1, an autosomal dominant monogenic form of diabetes, providing human genetic evidence for a direct role in diabetes pathogenesis. HNF4α gene expression is down-regulated in T2D. In addition, free fatty acids, which are elevated in overweight and obese individuals, inhibit HNF4α activity. In view of the fact that HNF4α haplo insufficiency causes diabetes and HNF4α is down-regulated in T2D, restoration of or an increase in HNF4α activity to the normal wild-type state would provide an overall health and therapeutic benefit.

HNF4α-knockout rodent models exhibit the fatty liver phenotype, as well as reduced lipogenesis, reduced de-novo cholesterol synthesis, reduced very-low-density lipoprotein (VLDL) secretion and high-density lipoprotein (HDL) biogenesis, as well as increased insulin intolerance. In addition, knockout mice show enhanced uptake of FFAs and reduced degradation via β-oxidation. This results in hypocholesterolemia, low blood triglyceride levels, and hepatic steatosis. All of this represents a significant dysregulation of lipid metabolism resulting from HNF4α deficiency (Yin, et al. (2011) Arterioscler. Thromb. Vase. Biol. 31(2) :328-336; Hayhurst, et al. (2001) Mal. Cell Biol. 21(4)1393-1403; Martinez-Jimenez (2010) Mol. Cell. Biol. 30 (3): 565-577). By comparison, increased expression of HNF4α in the liver mayincrease transcription of genes that promote hepatic FFA oxidation, ketogenesis, and very-low density lipoprotein (VLDL) secretion, as a means to deal with excess FFA accumulation (Martinez-Jimenez (2010) Mol. Cell. Biol. 30(3) :565-577). Therefore, HNF4a provides a target for mitigating the adverse effects of FFAs, which are characteristically elevated in NAFLD.

In T2DM, HNF4α is responsible for direct regulation of genes involved in glucose transport and glycolysis. Without HNF4α in β-cells, rodents exhibit defective glucose-stimulated insulin secretion in β-cells—meaning decreased insulin secretion (Gupta, et al. (2005) J. Clin. Invest. 115(4)1006-15). It has been observed that HNF4α gene expression is downregulated in individuals with T2DM, likely due to exposure to chronically elevated FFAs. In particular, it has been shown that free palmitic acid (a C16 saturated FA) impairs pancreatic β-cell function and viability and suppresses normal insulin production due to actions on HNF4a (Lee, et al. (2013) ACS Chem. Biol. 8(8) :1730-1736). Therefore, HNF4α provides a target or ameliorating the symptoms of T2DM.

### Metabolic Disorders

The term “metabolic disorder” refers to a disorder or condition that occurs when the body is unable to properly metabolize carbohydrates, lipids, proteins, and/or nucleic acids. Accordingly, in the context of the present invention disorders relating to abnormality of metabolism are encompassed in the term “metabolic disorder.” The term metabolic disorder includes, but is not limited to, insulin resistance, hyperglycemia, diabetes mellitus (in particular T2DM), obesity, glucose intolerance, hypercholesterolemia, hyperlipoproteinemia, atherosclerotic disease, syndrome, hypertension, with abnormal plasma dyslipidemia, hyperinsulinemia, coronary artery disease, metabolic or a related disorder associated lipoprotein, triglycerides or a disorder related to glucose levels such as pancreatic beta cell regeneration.

T2DM refers to a chronic disease or condition, which occurs when the pancreas does not produce enough insulin, or when the body cannot effectively use the insulin it produces. This leads to an increased concentration of glucose in the blood (hyperglycemia). Based on studies that have established a relationship between plasma glucose concentrations, measures of glycemic exposure, and risk of diabetic retinopathy, the following criteria have been adopted for the diagnosis of diabetes mellitus: fasting plasma glucose greater than or equal to 126 mg/dL (7.0 mmol/L); plasma glucose greater than or equal to 200 mg/dL (11.1 mmol/L) at 2 hours following ingestion of 75 g anhydrous glucose in an oral glucose tolerance test; or random plasma glucose greater than 200 mg/dL (11.1 mmol/L) in a person with symptoms of diabetes. Other important definitions include: impaired glucose tolerance with a plasma glucose equal to or greater than 140 mg/dL (7.8 mmol/L) but less than 200 mg/dL (11.1 mmol/L) at 2 hours in the oral glucose tolerance test; and impaired fasting glucose with a fasting plasma glucose (FPG) equal to or greater than 100 mg/dL (5 6 mmol/L) but less than 126 mg/dL. A compound of the invention is said to modulate metabolism by decreasing one or more of fasting plasma glucose, plasma glucose following ingestion of 75 g anhydrous glucose, or random plasma glucose levels below those referenced herein. Another endpoint that can be monitored as part of assessment of metabolic activity is blood levels of HbAlC; HbAlc is a measure of average glucose levels in blood over the past two to three months. Levels of HbAlc are used as clinical indicators of risk of diabetes, where increased levels are indicative of an increased risk of T2DM. Thus, reduction in HbAlc can be used to support an indication of glycemic control.

Obesity is a chronic, relapsing health risk defined by excess body fat. Total body fat can be accurately measured using hydrodensitometry and dual-energy x-ray absorptiometry (DEXA). Because body mass index (BMI), expressed as kilograms of weight divided by height in meters squared (kg/m²), is simple and inexpensive to calculate, and correlates strongly with total body fat in non-elderly adults, it is commonly used as a surrogate for total body fat. Obesity is defined by the National Institutes of Health as having a BMI of 30 kg/m² or higher. The relationships between BMI and risks for death and major comorbidities vary by age, sex, race, and smoking status, but, in general, are lowest in individuals with BMIs of 18.5 kg/m² to 24.9 kg/m² and increase in a curvilinear or linear manner with BMIs of 25 kg/m 2 to approximately 40 kg/m 2 . A compound of the invention is said to modulate metabolism by decreasing mean and/or categorical body weight. Mean body weight is defined as the difference in mean percent loss of baseline body weight in the active product-treated versus placebo-treated group. Categorical body weight is defined as the proportion of subjects who lose at least 5 percent of baseline body weight in the active product-treated versus placebo-treated group. Secondary efficacy endpoints can include, but are not limited to, improvements in blood pressure and pulse, lipoprotein lipids, fasting glucose and insulin, HbAlc (in T2DM), waist circumference, and quality of life.

NAFLD, or “fatty liver,” is a metabolic disease characterized by excessive accumulation of fat in the liver. NAFLD macrovesicular is characterized steatosis and the by predominantly presence of visible steatosis in >5% of hepatocytes is generally accepted as a working definition of a fatty liver (Kleiner, et al. (2005) Hepatology 41:1313-1321). Nonalcoholic steatohepatitis or NASH is the most extreme form of NAFLD and is considered as a major cause of cirrhosis of liver of unknown etiology. The minimal criteria for the diagnosis of NASH include the presence of >5% macrovesicular steatosis, inflammation and liver cell ballooning, typically with a predominantly centrilobular (acinar zone 3) distribution in adults. Steatohepatitis is not simply the presence of inflammation and steatosis but is a specific histologic entity (Kleiner, et al. (2005) Hepatology 41(6):1313-21; Brunt, et al. (1999) Am. J. Gastroenterol. 94:2467-2474; Ludwig, et al. (1980) Mayo Clin. Proc. 55:434-438; Neuschwander-Tetri & Caldwell (2003) Hepatology 37:1202-1219). A compound of the invention is said to modulate metabolism by measurably reducing the accumulation of fat in the liver thereby improving liver function.

The term metabolic syndrome represents a cluster of laboratory and clinical findings that serve as markers for increased risk for coronary heart disease, stroke, peripheral vascular disease and/or T2DM. Risk factors associated with metabolic syndrome include abdominal obesity (i.e., excessive fat tissue in and around the abdomen) , atherogenic dyslipidemia including but not limited to high triglycerides, low HDL cholesterol and high LDL cholesterol, elevated blood pressure, insulin resistance or glucose intolerance, prothrombotic state (e.g., high fibrinogen or plasminogen activator inhibitor-1 in the blood), and/or proinflammatory state (e.g., elevated C-reactive protein in the blood). A compound of the invention is said to modulate metabolism by improving components of metabolic syndrome and ultimately shown to prevent the development T2DM and reduce cardiovascular morbidity and mortality.

### Metabolism Modulation

This invention also provides methods for modulating metabolism to ameliorate, prevent or treat a metabolic disorder. In accordance with such methods, an effective amount of a compound of this invention is administered to a subject in need of treatment so that the subject's metabolism is modulated thereby addressing the underlying pathogenesis of one or more metabolic disorders and promoting the health, well-being, and quality of life of the subject. The term “subject” as used herein refers to an animal, preferably a mammal. In some embodiments, the subject is a veterinary, companion, farm, laboratory or zoological animal. In other embodiments, the subject is a human.

A subject in need of treatment includes a subject with observable symptoms of a metabolic disorder (e.g., a subject with abnormal glucose or lipid metabolism), as well as a subject who has no observable symptoms of a metabolic disorder but has been determined to be susceptible to developing the metabolic disorder (i.e., a subject at risk of developing the metabolic disorder). For example, according to the American Heart Association, metabolic syndrome (which raises the risk of heart disease, diabetes, stroke, and other health problems) is diagnosed when any three of the following five risk factors are present: high blood glucose (sugar); low levels of HDL (“good”) cholesterol in the blood; high levels of triglycerides in the blood; large waist circumference or “apple-shaped” body; or high blood pressure.

By way of further illustration, autoantibodies to insulin (IAA); glutamic acid decarboxylase (GAD); and an islet cell member of the receptor type of the tyrosine phosphate family termed IA-2 have been identified as markers that predate the clinical onset of T2DM. See, e.g., U.S. Pat. Nos. 6,391,651 and 6,316,209. Similarly, C-reactive protein (CRP), apolipoprotein CIII, and plasma homocysteine levels have been identified as markers for identifying subjects at risk for high cholesterol (or hypercholesterolemia or hyperlipidemia) See, e.g., US 2004/0198656; Yeh (2004) Can. J. Cardiol. 20(Suppl B):93-96B; and Geisel, et al. (2003) Clin. Chem. Lab. Med. 41(11):1513-7. Additional factors that can be used, alone or in combination, to determine whether a subject is at risk or predisposed to developing hypercholesterolemia include, without limitation, heredity (i.e., familial hypercholesterolemia), high blood pressure, history of smoking, alcohol consumption, diabetes, obesity, physical inactivity, age and sex (i.e., post-menopausal women over the age of 50), and stress.

The term “effective amount” as used herein means an amount of the compound or formulation containing the compound, which is sufficient to significantly improve a disorder. Also of concern when determining an effective amount to be used in humans is balancing the desired effects (benefits) against risks associated with use of a compound. At issue for such risk/benefit assessments is the types of adverse effects observed and the likelihood that they will occur. Also considered is the fact that the effective amount may vary with the particular disorder being treated, e.g., diabetes mellitus or obesity, the age and physical condition of the end user, the severity of the condition, the duration of the treatment, the particular carrier utilized, and like factors.

In general, a suitable daily dose of a compound of the invention will be that amount of a compound which is the lowest dose that is effective at producing a desired benefit, in this case an effect that improves metabolism of fats and sugars and consequently supports overall health and well-being. Such an effective dose will generally depend upon the factors described herein. For oral administration, the dose may range from about 0.0001 mg to about 10 grams per kilogram of body weight per day, about 5 mg to about 5 grams per kilogram of body weight per day, about 10 to about 2 grams per kilogram of body weight per day, or any other suitable dose. If desired, the effective daily dose of the compound may be administered as two, three, four, five, six or more sub-doses administered separately at appropriate intervals throughout the day, optionally, in unit dosage forms. Preferred dosing is one administration per day.

The compound of the invention can be used alone or in combination with a particular diet (e.g., foods with a low glycemic index) or standard of care.

Administration of a compound of the invention modulates the metabolism of a subject thereby addressing the underlying pathogenesis of one or more metabolic disorders and/or promoting the health, well-being, and quality of life of the subject. Ideally, an effective amount of a compound provides a measurable improvement in the levels or activity of one or more metabolic compounds. Examples include HNF4a activity, insulin-like growth factor levels (such as insulin-like growth factor 1 or IGF-1), blood sugar levels, insulin levels, C peptide levels, triglyceride levels, free fatty acid levels, blood uric acid levels, microalbuminuria levels, glucose transporter expression, adiponectin levels, total serum cholesterol levels, high density lipoprotein (HDL) levels, and/or low density lipoprotein (LDL) levels.

More particularly, administration of a compound of the invention improves metabolism, liver function, fasting plasma glucose levels, postprandial plasma glucose levels, glycosylated hemoglobin HbAlc, body weight, insulin sensitivity, serum lipid profile by improving lipid clearance, or a combination thereof. In particular embodiments, use of a compound of the invention preferably prevents, slows the progression of, delays or treats a metabolic disorder such as T2DM, impaired glucose tolerance, impaired fasting blood glucose, hyperglycemia, postprandial hyperglycemia, hyperinsulinemia, NASH, NAFLD, or metabolic syndrome; slows the progression of, delays or treats pre-diabetes; improves glycemic control and/or reduces fasting plasma glucose, postprandial plasma glucose and/or glycosylated hemoglobin HbAlc; prevents, slows, delays or reverses progression of impaired glucose tolerance, impaired fasting blood glucose, insulin resistance or metabolic syndrome to T2DM; prevents, slows the progression of, delays, prevents or treats a complication of diabetes mellitus such as cataracts or a micro- or macrovascular disease, such as nephropathy, retinopathy, neuropathy, tissue ischemia, diabetic foot, dyslipidemia, arteriosclerosis, myocardial infarction, acute coronary syndrome, unstable angina pectoris, stable angina pectoris, stroke, peripheral arterial occlusive disease, cardiomyopathy, heart failure, heart rhythm disorders or vascular restenosis; reduces body weight and/or body fat, or prevents an increase in body weight and/or body fat, or facilitates a reduction in body weight and/or body fat; prevents, slows, delays or treats diseases or conditions attributed to abnormal accumulation of ectopic fat, in particular liver fat; maintains and/or improves insulin sensitivity and/or treats or prevents hyperinsulinemia and/or insulin resistance; reduces fat deposits; prevents, slows, delays or reverses progression of fatty liver to NASH; and/or prevents, slows, delays or reverses progression of NASH to cirrhosis, end-stage liver disease and/or hepatocellular carcinoma.

The following non-limiting examples are provided to further illustrate the present invention.

### Example 1: Assessing Indicators of Metabolic Activity

### Materials and Methods

Expression of Insulin and HNF4α. RNA was purified using a RNEASY® chromatographic separation and isolation kits (Qiagen), and converted to cDNA using the gScript™ cDNA SuperMix (Quanta Biosciences). Q-PCR was conducted with cDNA corresponding to 2 μg of RNA using an Opticon Real-Time System (MJ Research) and QPCR SuperMix (BioPioneer). See All mRNA values were normalized to 18S rRNA values and are expressed as fold changes over vehicle-treated control.

Counter-Screen for Estrogenic Activity. Estrogenic activity was monitored by co-transfection of a reporter plasmid containing a multimerized E-box 5′ of a minimal promoter fused to the Firefly luciferase gene with wild-type E47 or E47MER (Kiselyuk, et al. (2010) J. Biomol. Screen 15(6):663-70). HeLa cells were transfected using polyethylenimine, 0.2 μg 4RTK-Luc plasmid and either 0.3 μg of human E47, E47MER or pMSCVhph vector in 50 μl of serum-free Dulbecco's modification of Eagle medium per well. Transfections included Renilla luciferase (pRL-TK) plasmid as a control for transfection efficacy. Transfection conditions were as described in the PPRE-Luc reporter assay of Kiselyuk, et al. ((2010) J. Biomol. Screen 15(6):663-70). Sixteen hours after transfection, culture media were changed and maintained for 48 hours with tamoxifen and/or compound or vehicle (DMSO). Cells were then lysed and assayed for luciferase activity using the Promega DUAL-LUCIFERASE® reporter assay kit (Promega Corp., Madison, WI), and luminescence was measured using the Veritas™ Microplate Luminometer (Turner Biosystems, Sunnyvale, CA). Data were normalized to Renilla luciferase (pRL-TK) and expressed as fold-change over vehicle alone.

Inhibition of HNF4α GFP Expression. Using the insulin promoter assay described herein, activity of HNF4α was assessed in the presence of BI-6015 (0, 2.5, 5 μM), a known antagonist of HNF4α (Kiselyuk, et al. (2012) Chem. Biol. 19(7):806-818), in combination with N-trans-caffeoyltyramine (0, 5, 10, 20 μM).

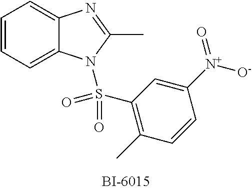 [SMILES: Cc1ccc([N+](=O)[O-])cc1S(=O)(=O)n1c(C)nc2ccccc21]

Hepatic Microsome Assay. Hepatic microsomes stability assays were performed in accordance with known methods (Peddibhotla, et al. (2013) ACS Med. Chem. Lett. 4:846-851). Briefly, 3 μL of 25 μM compound solution in acetonitrile were incubated with 123 μL of mouse, human or rat liver microsomes (Xenotech, Kansas City, KS). After preincubation at 37° C. for 10 minutes, enzyme reactions were initiated by adding 120 !at of NADPH-generating system (2 mM NADP+, 10 mM glucose-6-phosphate, 0.4 U/ml glucose-6-phosphate dehydrogenase, and 5 mM MgCl₂) in the presence of 100 mM potassium phosphate buffer (pH 7.4). The final concentration of each compound used was 1 μM. The microsomal concentrations used were 1.0 mg/mL. Compounds were incubated in microsomes for 0, 5, 15, 30 and 60 minutes. The reactions were stopped by the addition of ice cold ACN and the reaction mixtures were centrifuged at 10,000 g for 10 minutes before the supernatant was removed for analysis. A 10 μL portion of the resulting extract was injected on a Thermo HPLC system equipped with PAL CTC plate sampler (96-well plate), Dionex Ultimate 3000 binary pump (flow rate at 0. 600 mL/min), Dionex Ultimate 3000 thermostatted column compartment (temperature at 40° C.), Thermo Endura Mass Spectrometer (ESI source), using a Thermo Scientific Accucore C18 (2.6 μM, 2.1×50 mm) column. A gradient was run starting at 95% H₂O (0.1% formic acid) and 5% ACN (0. 1% formic acid) during the first 0. 5 min, then under gradient condition of 5-100% ACN (0.1% formic acid) from minute 0. 5 to 3.5, finishing at 95% H₂O (0.1% formic acid) and 5% ACN (0.1% formic acid) over 0.5 min, with another 1 min at 95:5 to re-equilibrate.

Lipid Clearance in HepG2 and T6PNE Cells (Steatosis Assay). The steatosis assay was carried out as described (Kiselyuk, et al. (2012) Chem. Biol. 19(7):806-818) with the exception of the drug concentration, which was 20 μM for N-trans-caffeoyltyramine with 0.25 mM palmitate in HepG2 cell lines, and 10 μM of either N-trans-caffeoyltyramine, N-trans-caffeoyltyramine, or p-coumaroyltyramine, with 0.25 mM palmitate in T6PNE cell lines. Steatosis was assessed using the Oil Red O Method for Fats kits (Poly Scientific; Warrington, PA), per manufacturer's guidelines. Briefly, frozen tissue slides or fixed cells were incubated in neat propylene glycol for 2 minutes and Oil Red O solution for 15 hours for slides or 1 hour for fixed cells, differentiated in 85% propylene glycol solution for 1 minute, washed twice with distilled water and stained in Hematoxylin of 10 seconds. Slides were mounted with glycerin jelly mounting medium.

Alkaline Phosphatase (ALP) Quantitation. Increased levels of ALP in blood are considered indicative of liver function abnormalities. Thus, ALP was assayed in accordance with known methods (Kiselyuk, et al. (2012) Chem. Biol. 19(7):806-818). Briefly, prior to sacrifice, blood was drawn and analyzed using a VetScan blood analyzer, measuring alkaline phosphatase (ALP, IU/L), alanine aminotransferase (ALT, IU/L), gamma glutamyl transferase (GGT, IU/L), bile acids (BA, μmol/L), total bilirubin (TBIL, mg/dL), albumin (ALB, g/dL), blood urea nitrogen (BUN, mg/dL), and cholesterol (CHOL, mg/dL).

Triglyceride (TG) Quantitation. TG quantity was assayed using a Triglyceride Colorimetric Assay Kit (Cayman Chemicals; Ann Arbor, MI) according to the manufacturer's instructions.

Lipid Droplet Size Analysis. All slides were scanned at a magnification of 20× using the Aperio Scanscope FL system (Aperio Technologies Inc.; Vista, CA). The appropriate dyes were assigned and illumination levels were calibrated using a preset procedure; the parameters were saved and applied to all slides. The acquired digital images represented whole tissue sections. Sections were evaluated for image quality. All acquired images were subsequently placed in dedicated project folders, and stored on a designated local server. Selected areas of the slides were selected using Aperio Imagescope (version 12 Aperio Technologies Inc.). For analysis, slides were viewed, whole tissue areas were selected and analyzed using the web-based Image Scope viewer. Slides were quantified using the ‘Color Deconvolution v9’ algorithm for oil redo staining (version 11 Aperio Technologies Inc.). The algorithm was optimized using a preset procedure to maximize the strong red color positive oil droplets signal to noise ratio and the subsequent macro was saved and applied to all slides.

HNF4α Immunostaining in Organ Samples. Samples were harvested from mice, fixed in 4% paraformaldehyde and embedded in paraffin or O.C.T. freezing media (Sakura Finetek; Torrance, CA). Slides of 5 μm thickness were washed four times with PBS and treated with 0.3% Triton™ in PBS for 10 minutes. Antigen retrieval was carried out with CitriSolv™ (Fisher Scientific; Waltham, MA) for 10 minutes in sub-boiling temperature. After washing with PBS for 10 minutes, slides were incubated in blocking solution with 5% normal donkey serum (Jackson Immuno Research; West Grove, PA) for 60 minutes at room temperature. Cells were fixed in 4% paraformaldehyde for 15 minutes on 4° C. and washed with PBS, treated with 0.3% Triton™ in PBS for 10 minutes and blocked as previously described for slide samples.

Primary Antibodies. HNF4α antibodies were used (#sc-6556, Santa Cruz Biotechnology; Santa Cruz, CA and #3113, Cell Signaling Technology; Danvers, MA). For fluorescent imaging, samples were incubated with ALEXA FLOUR® 488 green-fluorescent dye or Rhodamine labeled anti-mouse, rabbit or goat and nuclei were counterstained with DAPI (4′,6-diamidino-2-phenylindole). Controls using secondary antibodies alone were used to ensure specificity of immunostaining. Fluorescently labeled sections were analyzed with a conventional inverted microscope (Olympus, PlanFl 40x/0.60) or with a confocal microscope equipped with a krypton/argon laser.

Bioavailability Determinations. Male C57BL/6 mice were administered N-trans-caffeoyltyramine or N-trans-feruloyltyramine via IV, intraperitoneal or oral route (three mice for each route)(Table 1).

TABLE 1

| Route | Formulation | Dosage (mg/kg) |
|---|---|---|
| IV | 1 mg/mL in 75% PEG 300/25% | 2.0 |
| | water, clear solution | |
| Oral | 3 mg/mL in 0.5% methyl | 30.0 |
| | cellulose, homogenous opaque | |
| | suspension with fine | |
| | particles | |
| IP | 3 mg/mL in 5% DMSO/5% | 30 |
| | Polysorbate 80/90% water, | |
| | clear solution | |

A blood sample from each mouse was drawn at 0.25, 0.5, 1, 2, 4, 6 and 24 hours after administration. An 8μL aliquot of blood was used for analysis. After adding 200 μL of an internal standard comprising 100 ng/mL Labetalol, 100 ng/mL dexamethasone, 100 ng/mL tolbutamide, 100 ng/mL Verapamil, 100 ng/mL Glyburide, and 100 ng/mL Celecoxib in ACN, the mixture was vortex-mixed and centrifuged at 12000 rpm for 15 minutes at 4° C. to pellet precipitated protein. Four μL at of the supernatant was injected for LC-MS/MS analysis. Bioavailability (%) was calculated using AUC_{0-inf}(% AUC_{Extra}<20%) or AUC₀₋ₗₐₛₜ, (% AUC_{Extra} >20%) with nominal dose.

pH Stability Assessment. Individual stock solutions were prepared in DMSO at concentrations of 10 mg/mL. Four different buffer solution s were prepared to achieve solutions with a pH of 2, 7.4, 8.5 and 10. For each pH assay, 5 μL of stock solution was added to 245 μL of buffered solution to a 2 mL tube, vortexed and incubated in a 37° C. water bath. At each timepoint, 50 μL aliquots were taken, neutralized and analyzed via HPLC analysis using a DAD detector at 280 nm. The fold change of the peak area at 280 nm was analyzed for the initial and final timepoint, 0.5 and 72 hours, respectively.

### Example 2: Assessing Compounds for Activity as HNF4α Agonists

Given the role of HNF4α in maintaining a health metabolism in humans, test compounds were screened for activity as HNF4α agonists (either direct or indirect effects). Using a known insulin promoter-reporter assay Kiselyuk and colleagues (2010. J. Biomol. Screen 15(6):663-70) screened a library of compounds for activity to promote insulin activation. They identified an insulin activator (Kiselyuk, et al. (2012) Chem. Biol. 19(7):806-18) and the compound was subsequently shown to possess HNF4α agonistic activity in an ornithine transcarbamoylase (OTC) promoter assay. The OTC promoter is known to be responsive to HNF4α in transient transfection assays (Inoue, et al. (2002) J. Biol. Chem. 277:25257-65).

To identify plant compounds that have similar bioactivity as this synthetic agent, a bioinformatics approach was taken to predict, from the set of all known plant compounds, a targeted sub-set with the desired HNF4α agonistic activity. Using a number of algorithms in combination with training data (i.e., positive data), models were built around important features of the positive data, which were predictive of the desired biological activity. More specifically, a set of 18 synthetic compounds with known ability to affect HNF4α activity were included in the positive data set. These structures were used to search a database of plant compounds for chemical structures that had similar structural features. of metrics were used to measure similarity. A number based on concepts from the fields of graph theory and information theory, either solely or in combination.

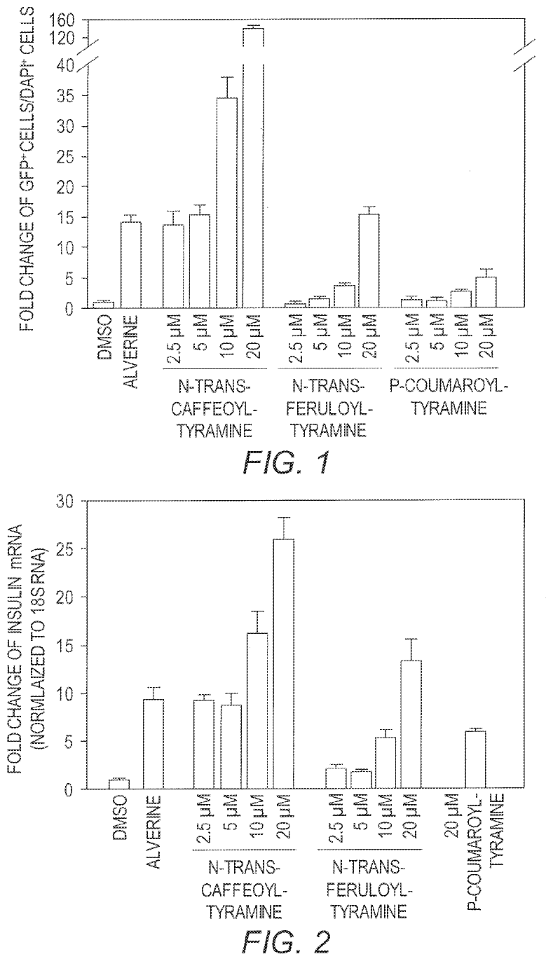

Plant compounds in the top 10th percentile of similarity to the 18 target structures were selected and compounds predicted to be potential agonists of HNF4α activity given their chemical structural features were screened in the HNF4α assay. The results of the screening identified a class of plant tyramine containing hydroxycinnamic acid amides (i.e., N-trans-caffeoyltyramine, N-cis-caffeoyltyramine, N-trans-feruloyltyramine and p-coumaroyltyramine) that are able to act as HNF4α modulators. Notably, N-trans-caffeoyltyramine was determined to be roughly an order-of-magnitude more potent than Alverine in activating HNF4α (FIG. 1) Due to hydroxyl derivatization of both phenyl rings, N-trans-caffeoyltyramine is less lipophilic and therefore expected to be more bioavailable. Overall, the increased potency and expected enhanced bioavailability indicated that N-trans-caffeotyramine and other tyramine containing hydroxycinnamic acid amides would be expected to be more desirable compounds for use in the methods disclosed herein.

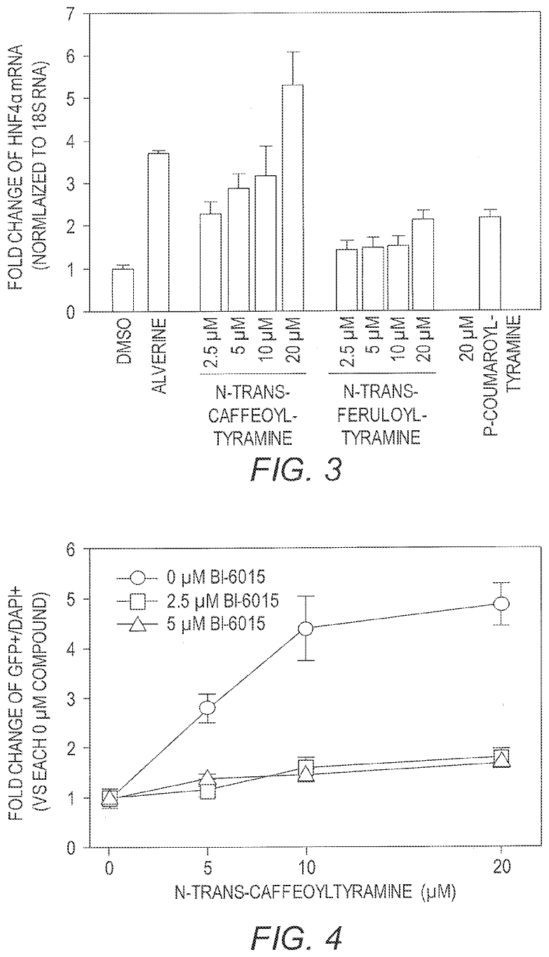

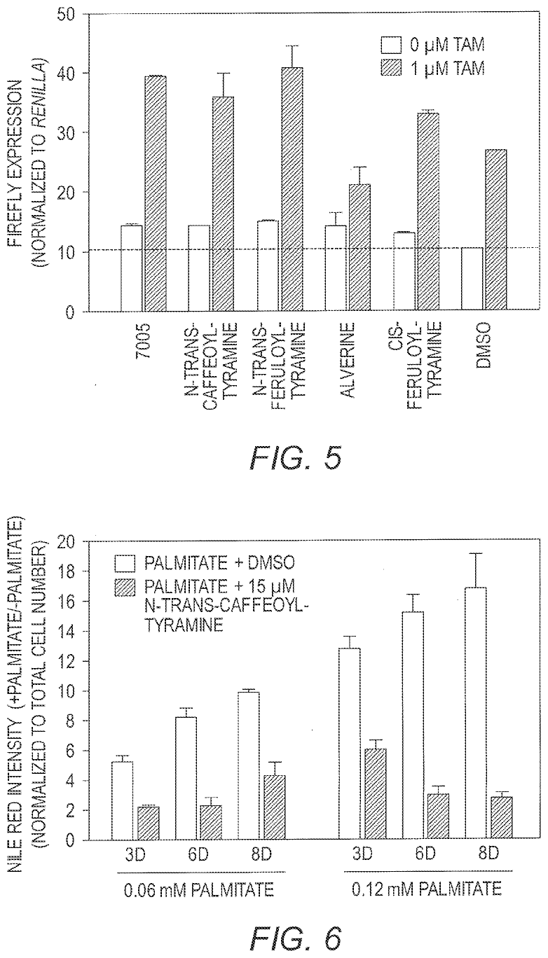

Secondary experiments were performed to demonstrate that these compounds directly modulate HNF4α activity. In particular, it was demonstrated that insulin (FIG. 2) and HNF4α (FIG. 3) gene expression were upregulated (e.g., as determined by quantitative PCR analysis) in the presence of N-trans-caffeoyltyramine and N-trans-feruloyltyramine. In addition, it was found that p-coumaroyltyramine also upregulated insulin and HNF4α gene expression however, cis-feruloyltyramine, N-coumaroyldopamine, N-trans-feruloyloctopamine and p-coumaroyloctopamine were inactive. Further, using the insulin promoter assay, N-trans-caffeoyltyramine-mediated increases in insulin expression were inhibited by BI-6015, a known HNF4α antagonist (FIG. 4). In addition, it was shown that N-trans-caffeoyltyramine and N-trans-feruloyltyramine did not exhibit estrogenic activity (FIG. 5).

Using human, rat and mouse hepatic microsomes, in vitro pharmacology indicated that N-trans-caffeoyltyramine was stable and that higher bioactivity in humans could be attributed to the longer half-life of N-trans-caffeoyltyramine in human cells compared to mouse hepatic microsomes (Table 2). For human microsomes, the apparent major biotransformation pathway was oxidation of the left-hand aryl ring.

TABLE 2

| | | | | Clearance | |
|---|---|---|---|---|---|
| | | | Half-life | Rate | % |
| Microsomes | Amount* | | (minutes) | (μl/min/mg) | Remaining |
| Mouse | 1 | μM | 0.8 | 1762.2 | 0.4 |
| | 10 | μM | 6.9 | 200.4 | 0.3 |
| Rat | 1 | μM | 2.1 | 674.2 | 0.6 |
| | 10 | μM | 22.4 | 30.9 | 33.6 |
| Human | 1 | μM | 77.3 | 17.9 | 55.3 |
| | 10 | μM | 262.3 | 5.3 | 85.4 |

*Amount of N-trans-caffeoyltyramine.

Analysis of HepG2 liver cells treated with N-trans-caffeoyltyramine (20 μM) or N-trans-feruloyltyramine (20 μM) indicated that these compounds were capable of clearing harmful fats from the liver, as evidenced by Oil Red 0 staining for fats, and further inhibited accumulation of fats in HepG2 liver cells treated with 0.25 mM palmitate. A similar inhibition of fat accumulation was observed in T6PNE cells treated with 0.25 mM palmitate and 10 μM N-trans-caffeoyltyramine, 10 1 μM N-trans-feruloyltyramine or 10 μM p-coumaroyltyramine N-trans-caffeoyltyramine reduced lipid accumulation when palmitate was added prior to compound administration (FIG. 6).

In addition to performing assays demonstrating beneficial effects of the compounds of the present invention, initial safety/toxicity assays were performed. The collective results of these analyses are presented in Table 3.

TABLE 3

| | N-trans- | N-trans- | |
|---|---|---|---|
| | caffeoyl | feruloyl | p-coumaroyl |
| Assay | tyramine | tyramine | tyramine |
| HNF4α Activity | + | + | + |
| HNF4α mRNA | + | + | + |
| Insulin mRNA | + | + | + |
| Estrogenic Counter- | + | + | + |
| Screen | | | |
| Fat Clearance | + | + | ND |
| pH Stability | Acid | Stable | Acid |
| | Stable | | Stable |
| Bioavailability | ~11% | ~7% | ND |

ND, not determined.

### Example 3: Efficacy in Diet-Induced Obese Mice

In addition to demonstrating in vitro efficacy of the compounds of the present invention, experiments were performed in vivo in animal models of human disease, i.e., diet-induced obese mice. The experiments were performed to establish feedingand treatment regimens, dosing and administration regimens, as well as to provide evidence of beneficial effects of N-trans-caffeoyltyramine on glucose and lipid homeostasis, hepatic steatosis, -cell function and hepatocyte function. Twelve mice (10 weeks old) were fed a high-fat diet for four weeks to induce obesity. After four weeks, and while on the high-fat diet, six mice were administered 5% DMSO or 120 mg/kg N-trans-caffeoyltyramine twice a day intraperitoneally for 14 days. One hour after the last i.p. injection of DMSO or N-trans-caffeoyltyramine, the animals were sacrificed and blood and organ (liver, kidney, gut and pancreas) samples were collected. Organ samples were subjected to histological, RNA, triglyceride and protein analyses. Notably, the mice in this study did not exhibit any toxic effects at any of the doses tested. The mice receiving treatment displayed levels of activity, alertness, grooming, and appetite consistent with the control group. None of the treated mice exhibited weight loss, sickness, or abnormal behaviors compared to the control group.

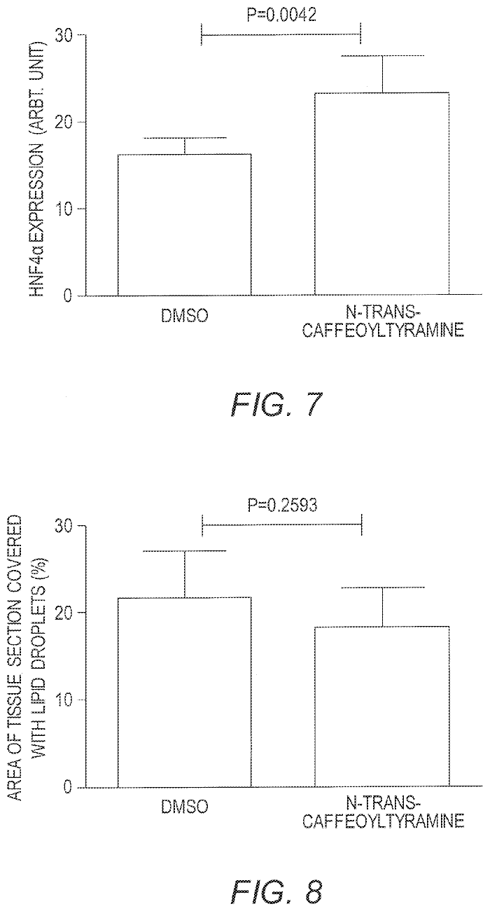

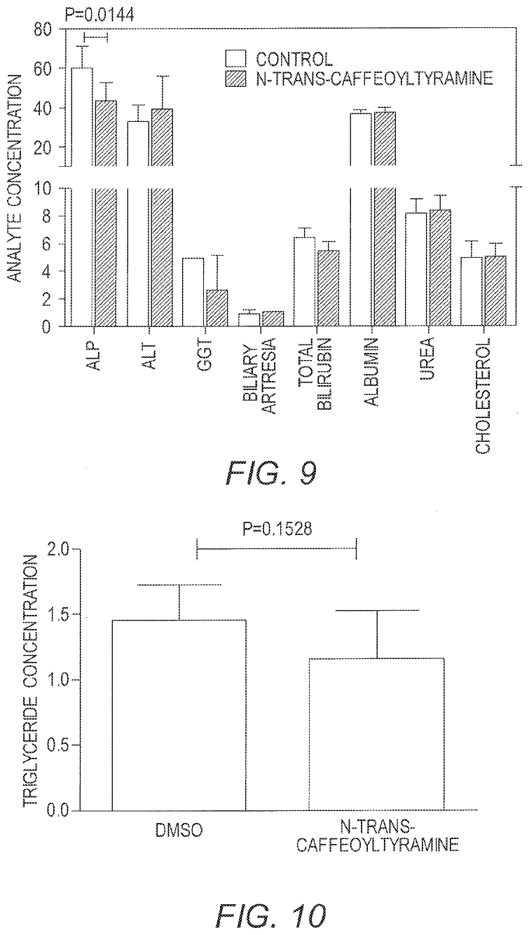

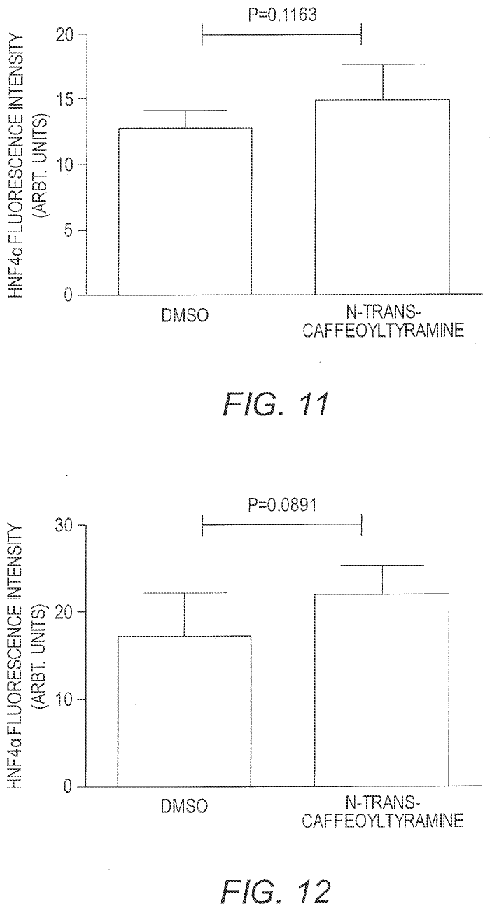

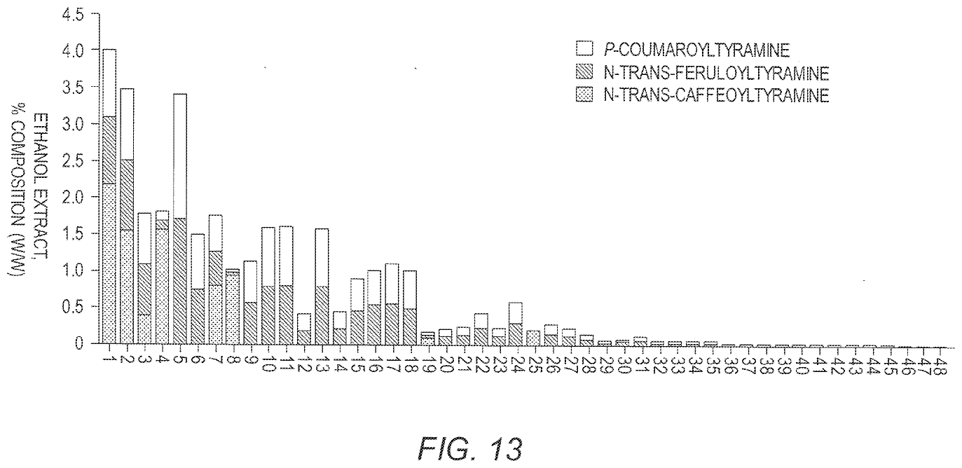

Results showed that N-trans-caffeoyltyramine treatment decreased lipid accumulation and significantly increased HNF4α expression (P=0.0042) in the liver, in particular nuclear expression of HNF4 α (FIG. 7) Immunostaining results indicated that N-trans-caffeoyltyramine increased HNF4 α activity. In addition, lipid droplet sizes in the liver were reduced in N-trans-caffeoyltyramine-treated animals (FIG. 8). In addition, levels of alkaline phosphatase (FIG. 9) and triglycerides (FIG. 10) were significantly reduced in mice treated with N-trans-caffeoyltyramine The reduction in liver fat and droplet size, and decrease in alkaline phosphatase demonstrate the beneficial effects of increasing HNF4A activity. Given that alkaline phosphatase and triglyceride levels are often a routine part of blood testing in humans, with elevated levels being an indication of poor liver functioning, obesity and metabolic syndrome, alkaline phosphatase and triglyceride levels would provide useful markers for assessing the effects of the tyramine containing hydroxycinnamic acid amides in humans administered compounds of the present invention. In the pancreas, HNF4 α expression was increased in N-trans-caffeoyltyramine-treated animals, as compared to DMSO control mice (FIG. 11) Similarly, HNF4 α expression was increased in intestines upon administration of N-trans-caffeoyltyramine (FIG. 12).

These in vivo data demonstrated a correlation between HNF4A expression and liver fat levels. In addition, results showed that N-trans-caffeoyltyramine increased HNF4A activity in vivo and produced beneficial effects on lipid, triglyceride, alkaline phosphatase and HNF4α levels.

### Example 4: Evaluation of Compound-Related Toxicity

Given the need to balance benefits and risks of the compounds of the present invention, in vivo toxicity studies in laboratory animals (e.g., mice, rats, dogs) are typically performed. Such studies are typically performed consistent with Good Laboratory Practice (GLP) regulations to ensure reliability and reproducibility for regulatory purposes. If compounds re to be administered for periods of weeks to months to years in humans, chronic toxicity studies typically are performed (studies of from six months to one year in duration) . For compounds to be used in foods, oral toxicity studies are recommended.

The purpose of a chronic toxicity study is to determine the toxicological profile of a test compound. In the initial phase of testing, a study will be performed in rats. A total of 160 Sprague Dawley rats (80 males and 80 females) approximately 5-7 weeks old and weighing between 80-100 g each will be randomly selected and allocated to treatment groups by weight; such that the mean body weights of each group will not be significantly different. The test compound will be administered orally at dose levels of 0.5, 1 and 2 g/kg body weight per day to rats for a period of 180 consecutive days. The animals will be observed daily for any clinical signs of toxicity (e.g., behavioral changes; skin and fur appearance; eating and drinking, etc.) as well as mortality. At the end of the experiment, the animals will be subjected to hematological, biochemical and histopathological evaluation consistent with standard toxicological methods.

### Example 5: Efficacy of Test Compounds in an Animal Model of NAFLD

Like the diet-induced obese mouse model, there are other well-established animal models for examining the benefits of compounds in NAFLD.

Animals and Diets. Adult male Sprague-Dawley rats (250-300 grams) will be obtained. Custom-prepared diets including control, High-fat only, and High-fat diet containing the test compound. Control diet will be a low-fat diet where 12% of total calories from fat is from corn oil, while most of the fat is linoleic acid. The High-fat (HF) diet will contain 60% of total calories as lard as well as 2% corn oil, and the diet will be enriched in oleic acid and the saturated fatty acids palmitic and stearic. such a High-fat diet was previously used to induce NAFLD in rats (Carmiel-Haggai, et al. (2005) FASEB J. 19:136-138). Seven rats in each of the 4 groups will be randomized and fed the diets for 4 weeks: Group I: control diet; Group II: HF diet, Group III: HF+0.5% compound diet; Group IV: HF+1% compound. The rats will be placed on a 12-hour day/night cycle and provided access to food and water ad libitum. At the end of 4 weeks, rats will be fasted 16-18 hours, anesthetized, and blood and liver samples will be collected for biochemical and histological analyses.

Serum and Liver Triglyceride and Cholesterol. Serum triglyceride and total cholesterol will be measured by commercially available assay kits (e.g., Wako Diagnostics; Richmond, VA). Total lipid will be extracted from liver samples (about 0.25 g) with chloroform-methanol mixture (2:1) and washed with 0.73% sodium chloride solution. The organic and aqueous phases will be separated by centrifugation at 2000 rpm for 10 minutes. The organic phase containing total lipid will be dried completely under nitrogen and lipid extract will be reconstituted in isopropanol. An aliquot of lipid extract will be used to measure triglycerides and total cholesterol using assay kits (e.g., from Wako Diagnostics).

Measurement of Serum and Liver Thiobarbituric Acid-Reactive Substances (TEARS). Serum and liver TBARS will be measured as an index of lipid peroxidation products.

Liver Histology. Liver samples will be fixed in 10% formalin and embedded in paraffin. Sections (5 μm) will be stained with hematoxylin and eosin and evaluated by a pathologist who will be blinded from the experimental groups and conditions. Sections will be subjected to semi-quantitation for assessing steatosis.

Statistical Analysis. Data will be presented as mean +S.E. Statistical analyses for the groups will be made using a two-tailed Student's t-test, and p<0.05 will be considered statistically significant.

### Example 6: An Evaluation of the Safety and Efficacy of Test Compounds in Treating NASH in Subjects with T2DM

The objective of the study will be to assess whether the test compounds of the present invention can improve liver health and liver fat content, as compared with placebo, in subjects who suffer from T2DM and NASH. The study also will include assessment of serum alanine aminotransferase (ALT) levels, and determination of whether test compounds are more effective than placebo treatment in reducing liver fat content (as measured by MRI-derived proton density-fat fraction, or MRI-PDFF). The comparison of serum ALT levels and liver fat content between test compound treatment and placebo treatment will be conducted in adult subjects with NASH and T2DM at week 24 (or the last post-baseline observation)

The secondary objectives of the study will be to evaluate the effects of the test compounds of the present invention, as compared with placebo treatment, on liver health. Endpoints monitored will include serum AST levels after 24 weeks of treatment; glycosylated hemoglobin (HbAlc) levels after 24 weeks; levels of liver fibrosis, as measured using transient Elastography with Fibroscan. Considered together, the results will allow for assessment of the safety and tolerability of test compound treatment as compared with placebo treatment.

Additionally, several exploratory objectives will be included in the study design. For example, the effect of the test compounds on the immune profile of subjects can be evaluated based on 1) a change from baseline in high-sensitivity c-reactive protein (hsCRP) and erythrocyte sedimentation rate (ESR); 2) a change from baseline in serum levels of tumor necrosis factor alpha (TNF-a); transforming growth factor (TGF) beta; 3) a change from baseline in levels of interleukin (IL)-2, -4, -6, -10, and -12; 4) a change from baseline in levels of interferon (IFN) gamma; and 5) an fluorescence-activated cell sorting (FACS) analysis which measures a change from baseline in immunological markers such as cluster of differentiation 3 (CD3), CD4, CDS, CD25, CD40, CD56, CD69, CD127, forkhead box P3 (FOXP3+), IL17, and retinoic acid-related orphan receptor-yt (RORyt)). Yet another exploratory objective will be to evaluate the effects of the test compounds on blood inflammatory markers (TNF- , fibroblast growth factor 19 (FGF-19)), liver fibrosis or cell death markers (cytokeratin-18 (CK-18), soluble Fas (sFas)), and oxidative stress markers such as hydroxyeicosatetraenoic acids (HETEs), hydroxyoctadecadienoic acids (HODEs), oxoeicosatetraenoic acids (oxoETEs), oxooctadecadienoic acids (oxoODEs) and ox-nonalcoholic steatohepatitis (ox-NASH) Moreover, the study will: evaluate the effect of the test compounds using the homeostatic model assessment of insulin resistance (HOMA IR) to measure insulin; evaluate the effect of the test compounds on serum lipid profile (triglycerides, high-density lipoprotein (HDL), low-density lipoprotein (LDL), and total cholesterol); and evaluate the effect of the test compounds on GLP1 and adiponectin.

Safety or tolerability endpoints will be evaluated after 24 weeks of treatment with the test compounds. Endpoints will include assessment of: the number and severity of any reported adverse events; physical examination findings; clinical laboratory evaluations (serum chemistry, hematology, and urinalysis) and 12-lead electrocardiograms (ECGs) from baseline to study completion; the number of subjects that withdraw from the study before completion of the protocol. The safety laboratory test results will be collected and measured at the following time points during the study: days -1 and 3 and weeks 1, 2, 3, 4, 6, 8, 10, 12, 14, 16, 18, 20, 22, and 24 (or early withdrawal).

A total of 80 T2DM and NASH subjects will be randomized into two groups: one group will receive a placebo, once daily (n=40); and one group will receive a dose of the test compound, 80 mg, once daily (n=40)

Although test compounds are planned to be administered at a dose of 80 mg per day, the dose may be titrated based on subject tolerability, or it may be set at a fixed amount for the duration of the study, regardless of tolerability.

### Example 7: An Evaluation of the Safety and Efficacy of Test Compounds in Treating NASH in Obese Subjects

A study will be conducted according to the methods of Example 6, wherein the only difference will be that the subject inclusion criteria include the requirement that the subjects are obese, as defined as having a BMI of 30 instead of T2DM.

### Example 8: An Evaluation of the Safety and Efficacy of Test Compounds in Treating NAFLD in Subjects with T2DM

The purpose of this study will be to determine whether the test compounds can improve liver fat content and liver health, as compared with placebo, in subjects who suffer from both T2DM and NAFLD by assessing magnetic resonance imaging-derived proton density fat fraction (MRI-PDFF) after 24 weeks of treatment.

The secondary objectives of this study will be: 1) to evaluate the effects of test compound treatment, as compared with placebo treatment, on liver health by assessing serum ALT levels after 24 weeks of treatment; 2) to evaluate the effects of test compound treatment, as compared with placebo treatment, on liver heath by assessing serum AST levels after 24 weeks of treatment; 3) to evaluate the effects of test glycosylated hemoglobin (HbAlc); 4) compound treatment on to evaluate the effects of test compound treatment on liver fibrosis, as measured using transient Elastography with Fibroscan; and 5) to evaluate the overall safety and tolerability of test compound treatment as compared with placebo treatment. Exploratory objectives of this study include those listed in Example 6.

A total of 80 T2DM and NAFLD subjects will be randomized into two groups: one group will receive a placebo, once daily (n=40) and one group will receive a dose of test compound, 80 mg, once daily (n=40) as described in Example 6. Subjects will be screened at visit 1 between days -28 and -2. At screening, subjects will undergo screening procedures meant to ensure that inclusion/exclusion criteria are met, including an abdominal MRI to quantitatively measure liver fat content. Subjects who meet inclusion/exclusion criteria based on the results of screening assessments will return to the study center on day -1 to undergo baseline assessments (visit 2). At the baseline visit, confirmation of inclusion/exclusion criteria will be performed, and assessments of baseline laboratory values, physical examination findings, and ECG results also will be performed.

Subjects will be required to have a certified histology report which documents and assesses the degree of steatosis, lobular inflammation, hepatocyte ballooning, and fibrosis that confirms a diagnosis of NAFLD.

At visit 18 on week 24 (or at early termination), all subjects will undergo end of treatment assessments, including liver fat content imaging by MRI and clinical laboratory safety assessments.

### Example 9: Derivatives of Tyramine Containing Hydroxycinnamic Acid Amide Derivatives

It is known in the art that Alverine and Benfluorex confer a structural change when binding to HNF4α (Lee, et al. (2013) ACS Chem. Biol. 8(8) :1730-1736.) Evaluation of both structures in the context of binding to HNF4α shows that both groups possess aliphatic aromatics on both ends with a central amine that can act as strong hydrogen bond acceptor. Using Alverine as the structural basis of bioactivity would suggest that N-2-phenylethylcinnamide, which possesses more structural similarity to Alverine and Benfluorex, would in theory be more likely to activate HNF4α in comparison to the hydroxylated analogs N-trans-caffeoyltyramine and N-trans-feruloyltryamine. However, the results presented herein demonstrate that the opposite effect is true, and that the position and methylation of the hydroxyls are critical to potency.

The structure of N-trans-caffeoyltyramine is as follows:

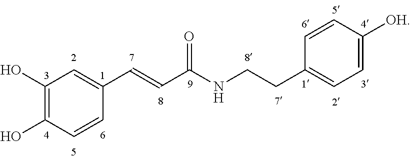 [SMILES: O=C(/C=C/c1ccc(O)c(O)c1)NCCc1ccc(O)cc1]

A hydroxy at the para position of each aromatic was found to maintain bioactivity for tyramine containing hydroxycinnamic acid amides. Loss of those hydroxyls (positions C4, C4′) as seen with N-2-phenylethylcinnamide and 1,7-diphenylhept-4-en-3-one leads to the complete loss of bioactivity. The hydroxy at the meta position of the C terminus (position C3) is also important though not critical to bioactivity. N-trans-feruloyltyramine demonstrates that loss of the hydrogen donating capacity of the meta hydroxy through methylation reduces but does not eradicate bioactivity, methylation removes hydrogen bond donating capabilities at this position but retains the capability for hydrogen bond accepting. Complete removal of the meta hydroxy (position C3) and its ability to act as a hydrogen bond acceptor or donor observed with N-trans-coumaroyltyramine confers an additional reduction but not eradication of bioactivity. Several other modifications lead to complete inactivity such as: addition of a hydroxyl to the methylene on the N terminus (position C7′) which converts the tyramine moiety to octopamine (N-trans-feruloyloctopamine). Isomerization of the double bond (bond C7-C8) to N-cis-feruloyltyramine, and addition of a hydroxyl to the meta position of the phenol (position C3′) on the N terminus (N-trans-coumaroyldopamine) all result in a complete loss of HNF4α activation.

Based on the screening results from over 60 pure compounds, structurally similar to N-trans-caffeoyltyramine, specific heteroatoms integral to bioactivity have been selected for modification for the structure-activity relationship (SAR) evaluation to improve upon the natural product and design a therapeutic that has strong oral bioavailability, pharmacokinetics (PK), Absorption, Distribution, Metabolism, and Excretion (ADME) and efficacious HNF4α activation.

The derivatives N-trans-caffeoyltyramine were designed to achieve one or more of:
(i) increasing or decreasing hydrophobicity (e.g., by addition/removal of branched or linear alkyl groups);
(ii) increasing or decreasing hydrophilicity (e.g., by addition/removal of charged or polar functional groups);
(iii) increasing enzymatic stability (e.g., by replacing amide bond with analogous bioisostere);
(iv) increasing chemical stability (e.g., by removal of alkene to eliminate potential isomerization);
(v) increasing or decreasing the pH of the molecule (e.g., by alkylating phenols to decrease acidity);
(vi) increasing potency or binding of the molecule to HNF4AQ (e.g., by optimizing substituent localization to increase binding affinity to the defined hydrophobic and hydrophilic pockets of the HNF4AQ binding site);
(vii) increasing hydrogen bond donation capabilities and locations (e.g., by addition of alcohols, carboxylic acids, tetrazole and amides);
(viii) increasing hydrogen bond acceptor capabilities and locations (e.g., by addition of alcohols, carboxylic acids, and amides or including 0, N and S heterocycles as well as their saturated analogs, which also add a hydrophilic nature to the molecule);
(ix) improving aqueous solubility (e.g., by addition of polar or charged groups);
(x) improving permeability properties; and/or
(xi) reducing toxicity.

In addition, the proposed modifications may slow clearance by inhibiting metabolism occurring through bond cleavage, heteroatom insertion or by glucuronidation giving the derivative a longer residence time.

Derivatives with Increased Hydrophilicity, Hydrophobicity, Hydrogen Bonding and Donating as well as pH Changes: Modification of Para Hydroxy Group of Tyramine.

The following compounds of Formula X provide derivatives with increased hydrophilicity, hydrophobicity, hydrogen bonding and donating as well as pH changes.

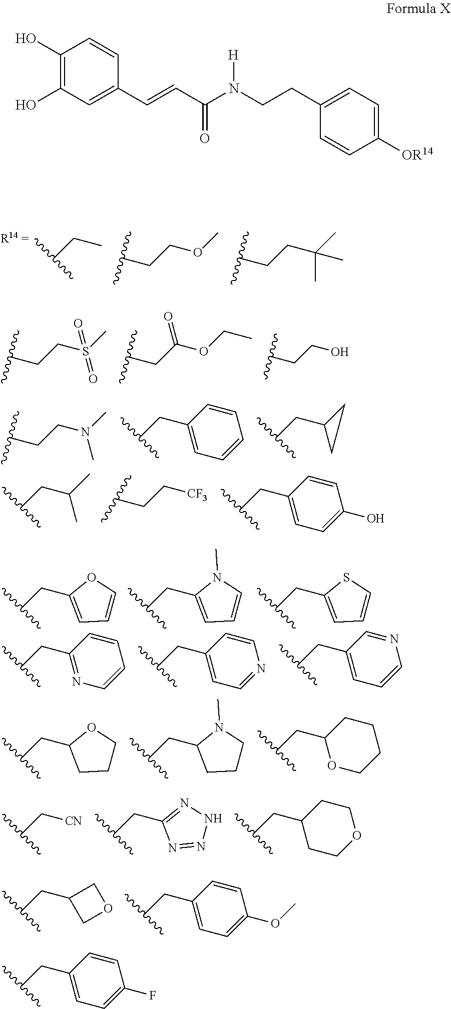

Synthesis of compounds of Formula X can be achieved by the method presented in Scheme 1.

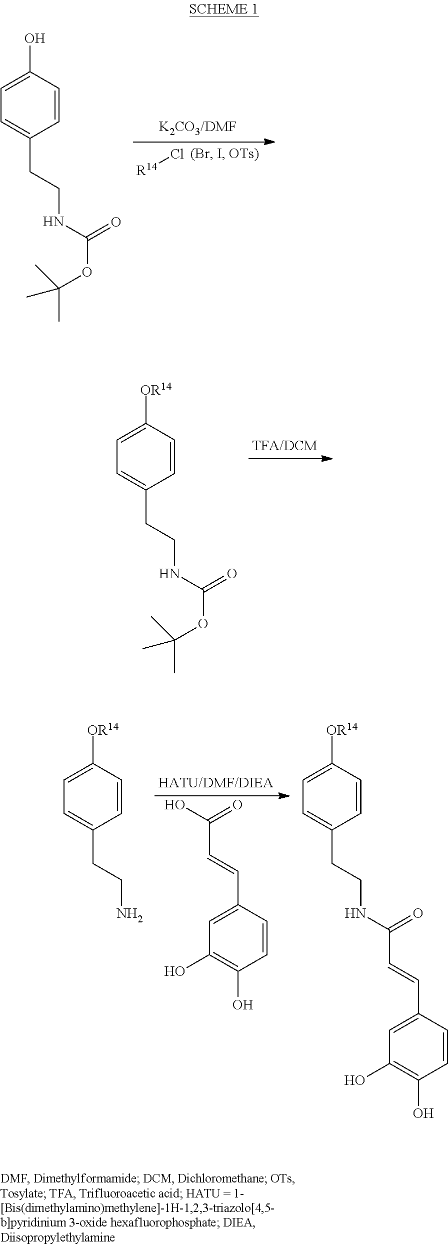

Derivatives with Increased Hydrophilicity, Hydrophobicity, Hydrogen Bonding and Donating as well as pH Changes: Modification of Meta Hydroxy Group of Cinnamoyl. The following compounds of Formula XI provide derivatives with increased hydrophilicity, hydrophobicity, hydrogen binding and donating as well as pH changes.

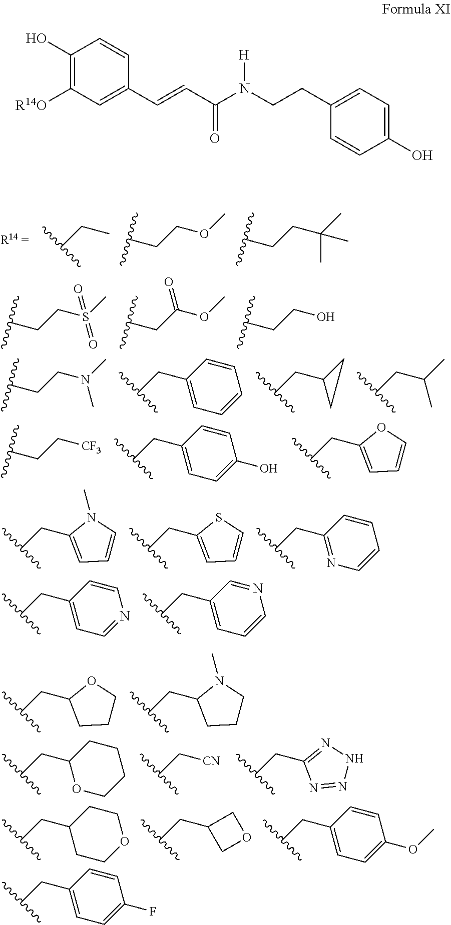

Synthesis of componds of Formula XI can be achieved by the method presented in Scheme 2.

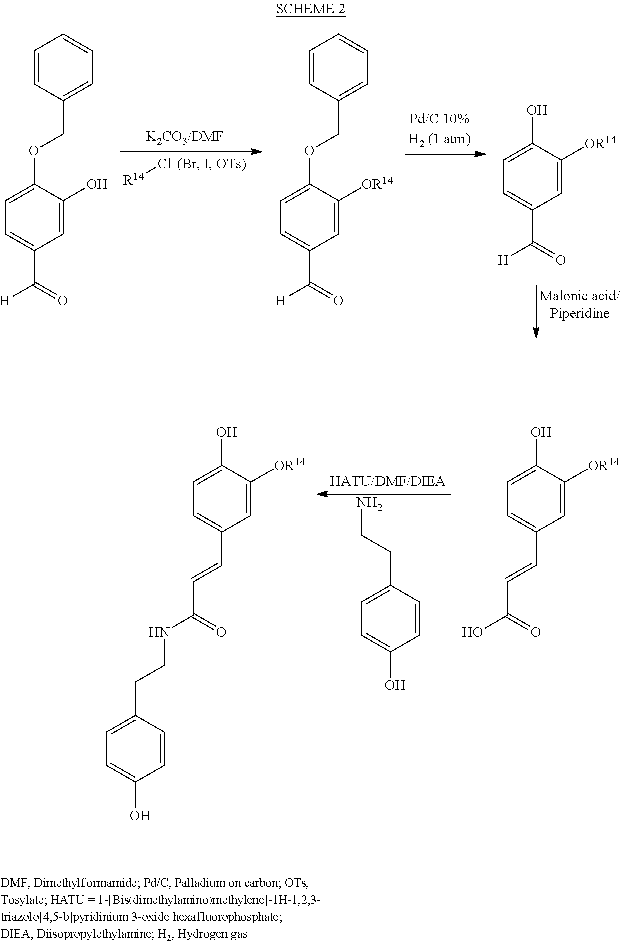

Derivatives with Increased Hydrophilicity, Hydrophobicity, Hydrogen Bonding and Donating as well as pH Changes: Modification of Para Hydroxy Group of Cinnamoyl.

The following compounds of Formula XII provide derivatives with increased hydrophilicity, hydrophobicity, hydrogen bonding and donating as well as pH changes.

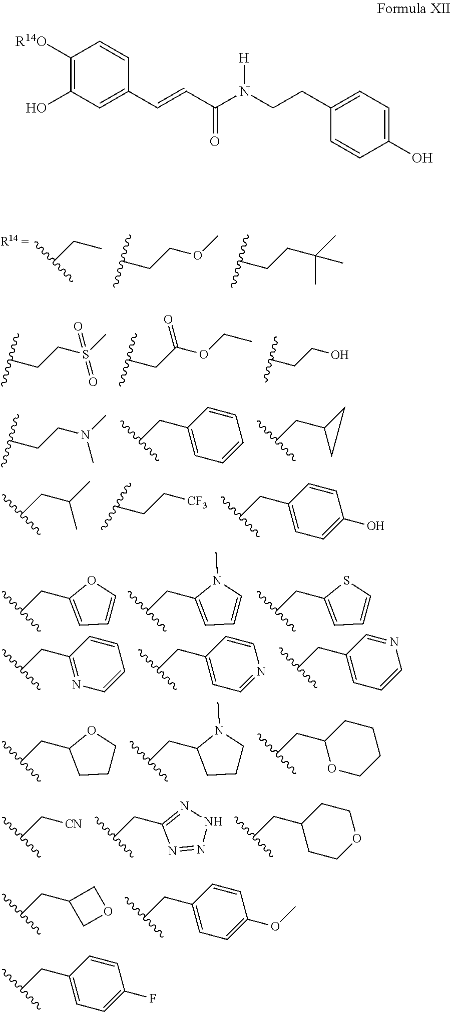

Synthesis of compounds of Formula XII can be achieved by the method presented in Scheme 3,

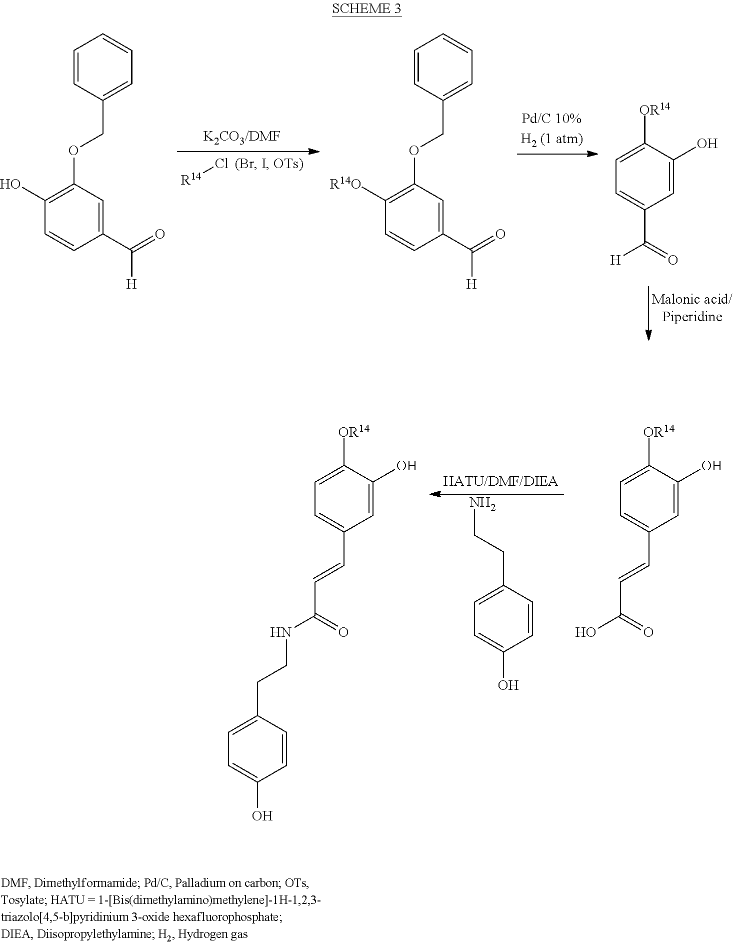

Derivatives with Increased Hydrophilicity, Hydrophobicity, Hydrogen Bonding and Donating as well as pH Changes: Modification of Nitrogen Group of Tyramine. The following compounds of Formula XIII provide derivatives with increased hydrophilicity, hydrophobicity, hydrogen binding and donating as well as pH changes.

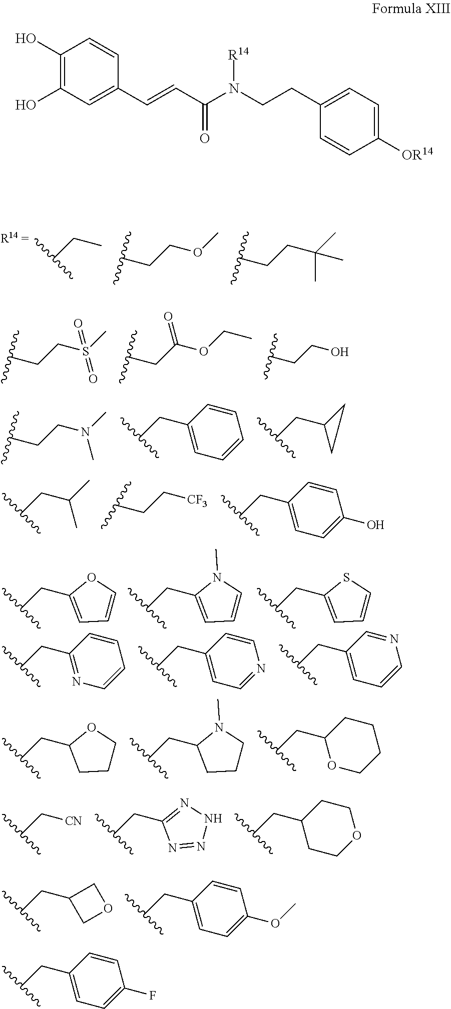

Synthesis of compounds of Formula XIII can be achieved by the method presented in Scheme 4.

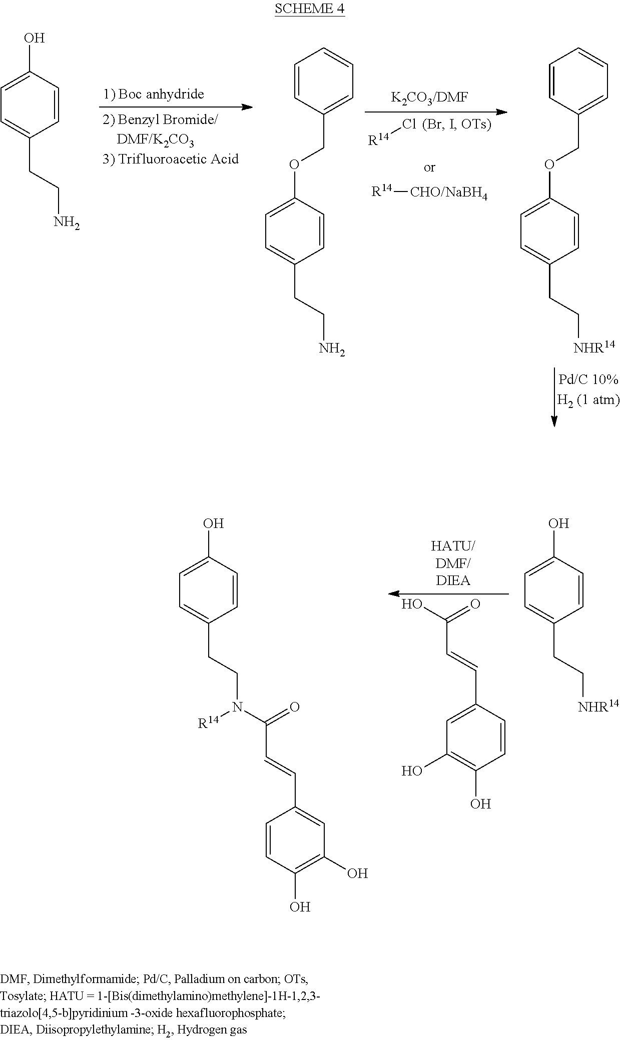

Additional Derivatives. In addition to those described above, the following derivatives are contemplated as exhibiting one or more improved characteristics compared to N-trans-caffeoyltyramine.

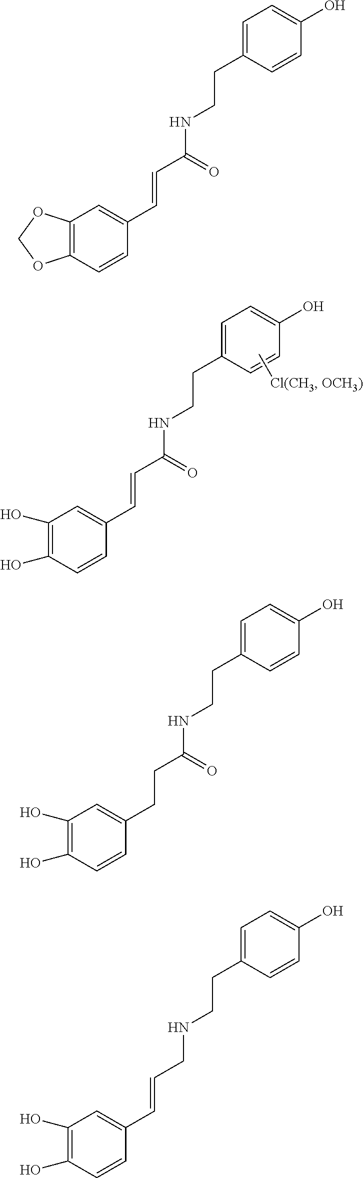

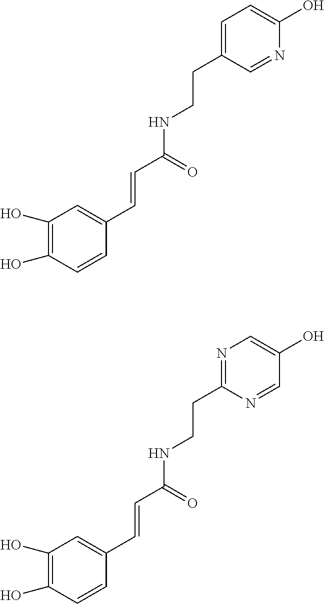 [SMILES: O=C(/C=C/c1ccc(O)c(O)c1)NCCc1ccc(O)nc1 ; O=C(/C=C/c1ccc(O)c(O)c1)NCCc1ncc(O)cn1]

Bioisosteres are of use in reducing metabolism and increasing selectivity of the active compound. Amide bioisosteres of N-trans-caffeoyltyramine include the following.

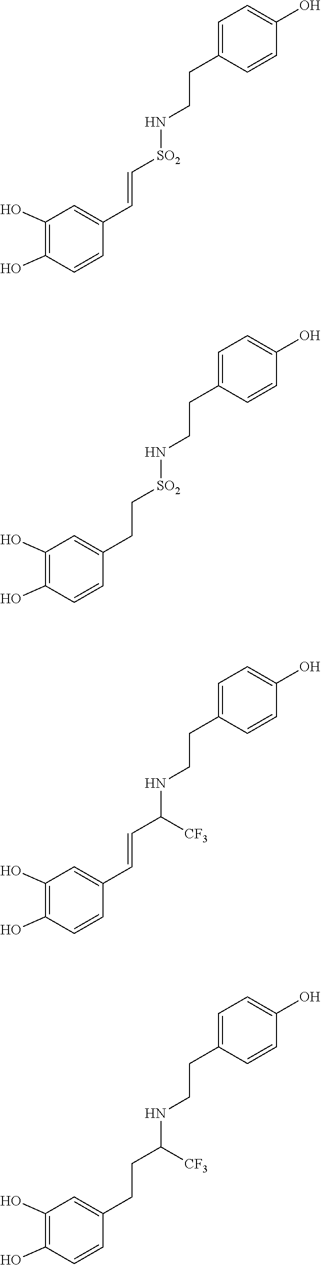 [SMILES: O=S(=O)(/C=C/c1ccc(O)c(O)c1)NCCc1ccc(O)cc1 ; O=S(=O)(CCc1ccc(O)c(O)c1)NCCc1ccc(O)cc1 ; Oc1ccc(CCNC(/C=C/c2ccc(O)c(O)c2)C(F)(F)F)cc1 ; Oc1ccc(CCNC(CCc2ccc(O)c(O)c2)C(F)(F)F)cc1]

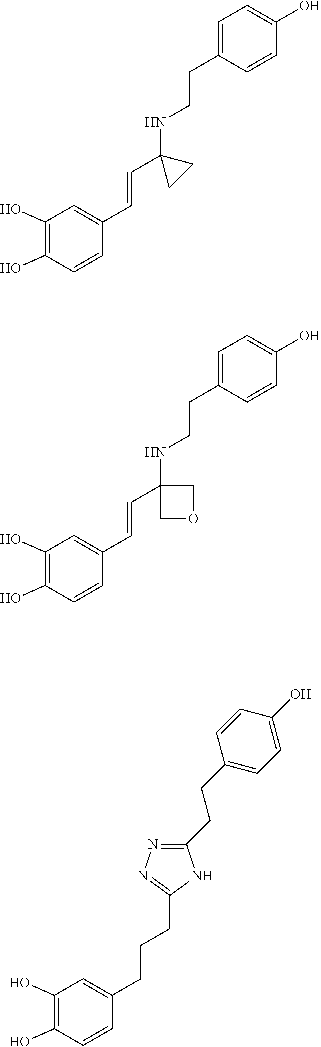 [SMILES: Oc1ccc(CCNC2(/C=C/c3ccc(O)c(O)c3)CC2)cc1 ; Oc1ccc(CCNC2(/C=C/c3ccc(O)c(O)c3)COC2)cc1 ; Oc1ccc(CCc2nnc(CCCc3ccc(O)c(O)c3)[nH]2)cc1]

Phenol bioisosteres of N-trans-caffeoyltyramine include the following.

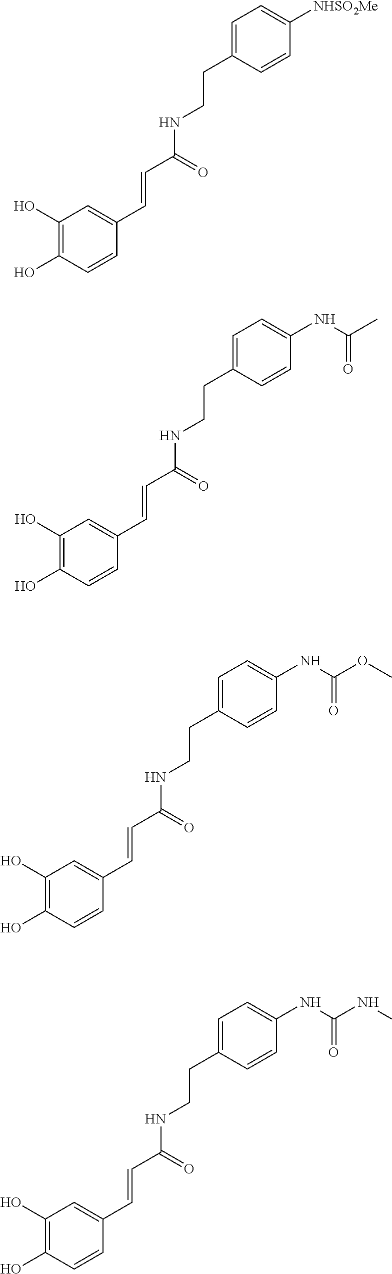

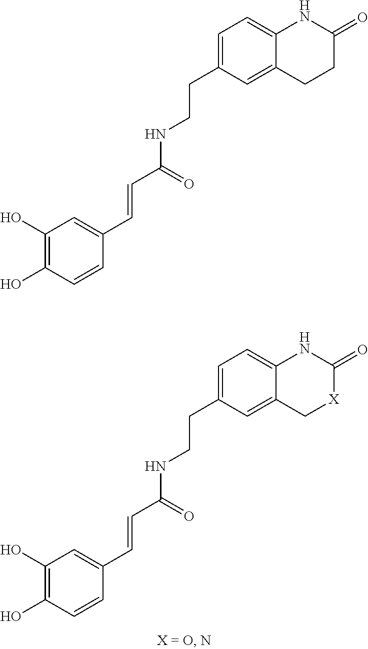

What is claimed is:

1. An orally consumable composition comprising
at least one carrier
a hemp hull derived N-trans-caffeoyltyramine; and
a hemp hull derived N-trans-feruloyltyramine,
wherein, the oral composition is in the form of a powder or granule.

2. The orally consumable composition of claim 1, wherein the hemp hull derived N-trans-caffeoyltyramine or hemp hull derived N-trans-feruloyltyramine is in the form of a pharmaceutically acceptable salt.

3. The orally consumable composition of claim 1, wherein the orally consumable composition is in a suitable form selected from the group comprising a dietary supplement, food ingredient, food additive, medical food, a nutraceutical, or pharmaceutical composition.

4. The orally consumable composition of claim 3, wherein the orally consumable composition is selected from the group comprising butter, margarine, sweet or savory spreads, condiment, biscuits, health bar, bread, cake cereal, candy, confectionery, soup, milk, yogurt, or a fermented milk product, cheese, juice-based beverage, vegetable-based beverages, fermented beverages, shakes, flavored waters, tea, or oil.

5. The orally consumable composition of claim 1, wherein the at least one carrier is selected from the group consisting of at least one of a sugar, a starch, or cellulose, powdered tragacanth, malt, gelatin, talc, excipient, oil, glycol, polyol, ester, agar, buffering agent, alginic acid, isotonic saline, ethyl alcohol, pH buffered solutions, or polyesters.

6. The orally consumable composition of claim 5, wherein the at least one carrier is cellulose and its derivatives.

7. The orally consumable composition of claim 6, wherein the cellulose and its derivatives is carboxymethylcellulose or methylcellulose.

8. The orally consumable composition of claim 1, wherein the pH of the composition is between 2 and 7.4.

9. The orally consumable composition of claim 3, wherein the orally consumable composition is in a unit dosage form and is configured for administration of less than 100 mg per day.

10. The orally consumable composition of claim 1, wherein the orally consumable composition provides between 10 and 60 mg/day of N-trans-caffeoyltyramine a day.

11. The orally consumable composition of claim 1, further comprising a preservative.

12. The orally consumable composition of claim 11, wherein the preservative is from about 0.01% to 1% by weight of the orally consumable composition.

13. The orally consumable composition of claim 1, wherein the hemp hull derived N-trans-caffeoyltyramine or hemp hull derived N-trans-feruloyltyramine is from an ethanol extract.

14. A dietary supplement, food ingredient or additive, a medical food, or nutraceutical for treating metabolic disorders, comprising,
a carrier;
a hemp hull derived N-trans-caffeoyltyramine; and
a hemp hull derived N-trans-feruloyltyramine,
wherein, the dietary supplement, food ingredient or additive, a medical food, or nutraceutical is in the form of a powder or granule.

15. The dietary supplement, food ingredient or additive, a medical food, or nutraceutical for treating metabolic disorders of claim 14, wherein the orally consumable composition is selected from the group comprising butter, margarine, sweet or savory spreads, condiment, biscuits, health bar, bread, cake cereal, candy, confectionery, soup, milk, yogurt, or a fermented milk product, cheese, juice-based beverage, vegetable-based beverages, fermented beverages, shakes, flavored waters, tea, or oil.

16. The dietary supplement, food ingredient or additive, a medical food, or nutraceutical for treating metabolic disorders of claim 14, wherein the at least one carrier is selected from the group consisting of at least one of a sugar, a starch, or cellulose, powdered tragacanth, malt, gelatin, talc, excipient, oil, glycol, polyol, ester, agar, buffering agent, alginic acid, isotonic saline, ethyl alcohol, pH buffered solutions, or polyesters.

17. The dietary supplement, food ingredient or additive, a medical food, or nutraceutical for treating metabolic disorders of claim 16, wherein the at least one carrier is cellulose and its derivatives.

18. The dietary supplement, food ingredient or additive, a medical food, or nutraceutical for treating metabolic disorders of claim 16, further comprising a preservative.

19. The dietary supplement, food ingredient or additive, a medical food, or nutraceutical for treating metabolic disorders of claim 18, wherein the preservative is from about 0.01% to 1% by weight of the orally consumable composition.

20. An orally consumable composition, comprising,
a carrier;
a preservative;
a hemp hull derived N-trans-caffeoyltyramine; and
a hemp hull derived N-trans-feruloyltyramine,
wherein, the orally consumable composition is in the form of a liquid.
